SEQUENCE LISTING (312 sequences)

SEQ ID NO: 1 - RNA, 19 nt, Artificial
  ZBTB7A
cagacaagaccuuaaauga

SEQ ID NO: 2 - RNA, 19 nt, Artificial
  ZBTB7A
guccgaugaugaccuggau

SEQ ID NO: 3 - RNA, 19 nt, Artificial
  ZBTB7A
gacaagcugaaggugcaca

SEQ ID NO: 4 - RNA, 19 nt, Artificial
  ZBTB7A
cucugagcggacguuaaaa

SEQ ID NO: 5 - RNA, 19 nt, Artificial
  ZBTB7A
gcagcuggaccuuguagau

SEQ ID NO: 6 - RNA, 19 nt, Artificial
  ZBTB7A
gcuggaccuuguagaucaa

SEQ ID NO: 7 - RNA, 19 nt, Artificial
  ZBTB7A
cacaucuucucgucucuuu

SEQ ID NO: 8 - RNA, 19 nt, Artificial
  ZBTB7A
cacugagacuucuugucaa

SEQ ID NO: 9 - RNA, 19 nt, Artificial
  ZBTB7A
ccucgcaauaaaaccaacu

SEQ ID NO: 10 - RNA, 19 nt, Artificial
  ZBTB7A
uguaacggaacggguacua

SEQ ID NO: 11 - RNA, 19 nt, Artificial
  ZBTB7A
caaauuccaaugucacaaa

SEQ ID NO: 12 - RNA, 19 nt, Artificial
  ZBTB7A
ccuuugcccacaacuacga

SEQ ID NO: 13 - RNA, 19 nt, Artificial
  ZBTB7A
cggacucgccuaaaaacca

SEQ ID NO: 14 - RNA, 19 nt, Artificial
  ZBTB7A
gaaucuaggguagcgcuuu

SEQ ID NO: 15 - RNA, 19 nt, Artificial
  ZBTB7A
gacauccugagugggcuga

SEQ ID NO: 16 - RNA, 19 nt, Artificial
  ZBTB7A
cugcacugagacuucuugu

SEQ ID NO: 17 - RNA, 19 nt, Artificial
  ZBTB7A
acaagcugaaggugcacau

SEQ ID NO: 18 - RNA, 19 nt, Artificial
  ZBTB7A
aggcacugacuguaaucca

SEQ ID NO: 19 - RNA, 19 nt, Artificial
  ZBTB7A
gacuacuaccugaaguacu

SEQ ID NO: 20 - DNA, 19 bp, Artificial
  ZBTB7A
gaaaacagaaacccgagaa

SEQ ID NO: 21 - RNA, 19 nt, Artificial
  ZBTB7A
cacugagacacaaaccuau

SEQ ID NO: 22 - RNA, 19 nt, Artificial
  ZBTB7A
cacaaauuccaaugucaca

SEQ ID NO: 23 - RNA, 19 nt, Artificial
  ZBTB7A
cggaacggguacuacacuu

SEQ ID NO: 24 - RNA, 19 nt, Artificial
  ZBTB7A
gcauaugcaaugcuagcau

SEQ ID NO: 25 - RNA, 19 nt, Artificial
  ZBTB7A
cgcaauaaaaccaacucua

SEQ ID NO: 26 - RNA, 19 nt, Artificial
  ZBTB7A
gguucugacgugaagaggu

SEQ ID NO: 27 - RNA, 19 nt, Artificial
  ZBTB7A
guuacaaccaacuucuaug

SEQ ID NO: 28 - RNA, 19 nt, Artificial
  ZBTB7A
auauauaugacgcgucaca

SEQ ID NO: 29 - RNA, 19 nt, Artificial
  ZBTB7A
gcccacaacuacgaccuga

SEQ ID NO: 30 - RNA, 19 nt, Artificial
  ZBTB7A
cugcacagacaccucaaga

SEQ ID NO: 31 - RNA, 19 nt, Artificial
  ZBTB7A
acuacuaccugaaguacuu

SEQ ID NO: 32 - RNA, 19 nt, Artificial
  ZBTB7A
cucugugacacacacaucu

SEQ ID NO: 33 - RNA, 19 nt, Artificial
  ZBTB7A
gcacugacuguaauccagg

SEQ ID NO: 34 - RNA, 19 nt, Artificial
  ZBTB7A
gcucugagcggacguuaaa

SEQ ID NO: 35 - RNA, 19 nt, Artificial
  ZBTB7A
cgguaacuucacagccgga

SEQ ID NO: 36 - RNA, 19 nt, Artificial
  ZBTB7A
cgccuaaaaaccaaaaaga

SEQ ID NO: 37 - RNA, 19 nt, Artificial
  ZBTB7A
cguuuggagauucaaaacu

SEQ ID NO: 38 - RNA, 19 nt, Artificial
  ZBTB7A
ggcaaccaaccacauuaga

SEQ ID NO: 39 - RNA, 19 nt, Artificial
  ZBTB7A
gaacggguacuacacuuua

SEQ ID NO: 40 - RNA, 19 nt, Artificial
  ZBTB7A
cggguacuacacuuuaucu

SEQ ID NO: 41 - RNA, 19 nt, Artificial
  ZBTB7A
caaaggcacugacuguaau

SEQ ID NO: 42 - RNA, 18 nt, Artificial
  ZBTB7A
guguacgagaucgacuuc

SEQ ID NO: 43 - RNA, 19 nt, Artificial
  ZBTB7A
guagaauccacuucuguuc

SEQ ID NO: 44 - RNA, 19 nt, Artificial
  ZBTB7A
gcugcacugagacuucuug

SEQ ID NO: 45 - RNA, 19 nt, Artificial
  ZBTB7A
ucaagaaagacggcugcaa

SEQ ID NO: 46 - RNA, 19 nt, Artificial
  ZBTB7A
ugcauaugcaaugcuagca

SEQ ID NO: 47 - RNA, 19 nt, Artificial
  ZBTB7A
ugagcccucuuuccaccuu

SEQ ID NO: 48 - RNA, 19 nt, Artificial
  ZBTB7A
acgagaucgacuucgucag

SEQ ID NO: 49 - RNA, 19 nt, Artificial
  ZBTB7A
cccacaacuacgaccugaa

SEQ ID NO: 50 - RNA, 19 nt, Artificial
  ZBTB7A
ggacucgccuaaaaaccaa

SEQ ID NO: 51 - RNA, 19 nt, Artificial
  ZBTB7A
ggguagcgcuuucucagau

SEQ ID NO: 52 - RNA, 19 nt, Artificial
  ZBTB7A
cuaugagucuuucagacaa

SEQ ID NO: 53 - RNA, 19 nt, Artificial
  ZBTB7A
ggagauucaaaacuucugu

SEQ ID NO: 54 - RNA, 19 nt, Artificial
  ZBTB7A
ccaaugucacaaaagcaau

SEQ ID NO: 55 - RNA, 19 nt, Artificial
  ZBTB7A
cguagaauccacuucuguu

SEQ ID NO: 56 - RNA, 19 nt, Artificial
  ZBTB7A
cuuuaaggacgaggacgag

SEQ ID NO: 57 - RNA, 19 nt, Artificial
  ZBTB7A
agaagaagauccgagccaa

SEQ ID NO: 58 - RNA, 19 nt, Artificial
  ZBTB7A
agaaucuaggguagcgcuu

SEQ ID NO: 59 - RNA, 19 nt, Artificial
  ZBTB7A
gguacuacacuuuaucuua

SEQ ID NO: 60 - RNA, 19 nt, Artificial
  ZBTB7A
cagggcacuuacuaaggga

SEQ ID NO: 61 - RNA, 19 nt, Artificial
  ZBTB7A
ggagaaaggagaucggacu

SEQ ID NO: 62 - RNA, 19 nt, Artificial
  ZBTB7A
cacauuagaagucuuggca

SEQ ID NO: 63 - RNA, 19 nt, Artificial
  ZBTB7A
cuuggcacuuuguaacgga

SEQ ID NO: 64 - RNA, 19 nt, Artificial
  ZBTB7A
cuuuguaacggaacgggua

SEQ ID NO: 65 - RNA, 19 nt, Artificial
  ZBTB7A
cucuuuuuacucaaaggca

SEQ ID NO: 66 - RNA, 19 nt, Artificial
  ZBTB7A
gguuuugguucccuucccu

SEQ ID NO: 67 - RNA, 19 nt, Artificial
  ZBTB7A
cuuucagacaagaccuuaa

SEQ ID NO: 68 - RNA, 19 nt, Artificial
  ZBTB7A
ucugugacacacacaucuu

SEQ ID NO: 69 - RNA, 19 nt, Artificial
  ZBTB7A
cugugacacacacaucuuc

SEQ ID NO: 70 - RNA, 19 nt, Artificial
  ZBTB7A
gcugcacugagacuucuug

SEQ ID NO: 71 - RNA, 19 nt, Artificial
  ZBTB7A
guuugcauaugcaaugcua

SEQ ID NO: 72 - RNA, 19 nt, Artificial
  ZBTB7A
acguguacgagaucgacuu

SEQ ID NO: 73 - RNA, 19 nt, Artificial
  ZBTB7A
agcuggaccuuguagauca

SEQ ID NO: 74 - RNA, 19 nt, Artificial
  ZBTB7A
ucccuccuuucugacguuu

SEQ ID NO: 75 - RNA, 19 nt, Artificial
  ZBTB7A
agagucacgaucagaggaa

SEQ ID NO: 76 - RNA, 19 nt, Artificial
  ZBTB7A
ccaacgugggugacauccu

SEQ ID NO: 77 - RNA, 19 nt, Artificial
  ZBTB7A
agggcacuuacuaagggag

SEQ ID NO: 78 - RNA, 19 nt, Artificial
  ZBTB7A
agccggacucgccuaaaaa

SEQ ID NO: 79 - RNA, 19 nt, Artificial
  ZBTB7A
aacggguacuacacuuuau

SEQ ID NO: 80 - RNA, 19 nt, Artificial
  ZBTB7A
caagaccuuaaaugauuuc

SEQ ID NO: 81 - RNA, 19 nt, Artificial
  ZBTB7A
ugcaagaccuucguccgcu

SEQ ID NO: 82 - RNA, 19 nt, Artificial
  ZBTB7A
gcacuuuaaggacgaggac

SEQ ID NO: 83 - RNA, 19 nt, Artificial
  ZBTB7A
gagaaaggagaucggacug

SEQ ID NO: 84 - RNA, 19 nt, Artificial
  ZBTB7A
gcacuuuguaacggaacgg

SEQ ID NO: 85 - RNA, 19 nt, Artificial
  ZBTB7A
acggaacggguacuacacu

SEQ ID NO: 86 - RNA, 19 nt, Artificial
  ZBTB7A
acguagaauccacuucugu

SEQ ID NO: 87 - RNA, 19 nt, Artificial
  ZBTB7A
cauuguuaaagggaagcuu

SEQ ID NO: 88 - RNA, 19 nt, Artificial
  ZBTB7A
acaucugcaagguccgcuu

SEQ ID NO: 89 - RNA, 19 nt, Artificial
  ZBTB7A
ugaaggugcacaugcggaa

SEQ ID NO: 90 - RNA, 19 nt, Artificial
  ZBTB7A
acuucugucuucguccucu

SEQ ID NO: 91 - RNA, 19 nt, Artificial
  ZBTB7A
aggcaacagugugggauaa

SEQ ID NO: 92 - RNA, 19 nt, Artificial
  ZBTB7A
uuagaagucuuggcacuuu

SEQ ID NO: 93 - RNA, 19 nt, Artificial
  ZBTB7A
acugagacuucuugucaau

SEQ ID NO: 94 - RNA, 19 nt, Artificial
  ZBTB7A
acaucuucucgucucuuuu

SEQ ID NO: 95 - RNA, 19 nt, Artificial
  ZBTB7A
cucaagaaagacggcugca

SEQ ID NO: 96 - RNA, 19 nt, Artificial
  ZBTB7A
ggcaacagugugggauaaa

SEQ ID NO: 97 - RNA, 19 nt, Artificial
  ZBTB7A
aaaaggcaaccaaccacau

SEQ ID NO: 98 - RNA, 19 nt, Artificial
  ZBTB7A
gcuugggccgguugaaugu

SEQ ID NO: 99 - RNA, 19 nt, Artificial
  ZBTB7A
uccucccuagcucagggau

SEQ ID NO: 100 - RNA, 19 nt, Artificial
  ZBTB7A
gcacagacaccucaagaaa

SEQ ID NO: 101 - RNA, 19 nt, Artificial
  YAP1
cagaagaucaaagcuacuu

SEQ ID NO: 102 - RNA, 19 nt, Artificial
  YAP1
gugcuaucauuagucacau

SEQ ID NO: 103 - RNA, 19 nt, Artificial
  YAP1
caggaauugagaacaauga

SEQ ID NO: 104 - RNA, 19 nt, Artificial
  YAP1
gugaguagguucauaaugu

SEQ ID NO: 105 - RNA, 19 nt, Artificial
  YAP1
gaacaaaacgagcaugaau

SEQ ID NO: 106 - RNA, 19 nt, Artificial
  YAP1
cucagacuuagaagucaga

SEQ ID NO: 107 - RNA, 19 nt, Artificial
  YAP1
cucuucaacgccgucauga

SEQ ID NO: 108 - RNA, 19 nt, Artificial
  YAP1
gaguacagacaguggacua

SEQ ID NO: 109 - RNA, 19 nt, Artificial
  YAP1
gaauugugggugugccuau

SEQ ID NO: 110 - RNA, 19 nt, Artificial
  YAP1
cuuggaaggagugccuaua

SEQ ID NO: 111 - RNA, 19 nt, Artificial
  YAP1
guagccacagauuaagauu

SEQ ID NO: 112 - RNA, 19 nt, Artificial
  YAP1
cgagaugagaguacagaca

SEQ ID NO: 113 - RNA, 19 nt, Artificial
  YAP1
gguuuaccuucauuuagcu

SEQ ID NO: 114 - RNA, 19 nt, Artificial
  YAP1
cagauggaguuuuagagua

SEQ ID NO: 115 - RNA, 19 nt, Artificial
  YAP1
gagaugagaguacagacag

SEQ ID NO: 116 - RNA, 19 nt, Artificial
  YAP1
gaguucugacauccuuaau

SEQ ID NO: 117 - RNA, 19 nt, Artificial
  YAP1
gagauggauacaggugaua

SEQ ID NO: 118 - RNA, 19 nt, Artificial
  YAP1
gcugccaccaagcuagaua

SEQ ID NO: 119 - RNA, 19 nt, Artificial
  YAP1
guacuuucagugcucaaaa

SEQ ID NO: 120 - RNA, 19 nt, Artificial
  YAP1
ccucgcaagcauguuguua

SEQ ID NO: 121 - RNA, 19 nt, Artificial
  YAP1
agaugagaguacagacagu

SEQ ID NO: 122 - RNA, 19 nt, Artificial
  YAP1
gagauggaaugaacauaga

SEQ ID NO: 123 - RNA, 19 nt, Artificial
  YAP1
gacagucuucuuuugagau

SEQ ID NO: 124 - RNA, 19 nt, Artificial
  YAP1
gacaguggacuaagcauga

SEQ ID NO: 125 - RNA, 19 nt, Artificial
  YAP1
guuguuucuucagcuuccu

SEQ ID NO: 126 - RNA, 19 nt, Artificial
  YAP1
cgagcaugaauuaacucuu

SEQ ID NO: 127 - RNA, 19 nt, Artificial
  YAP1
cugugauaccuggcacagu

SEQ ID NO: 128 - RNA, 19 nt, Artificial
  YAP1
ggagaccuaagaguccuuu

SEQ ID NO: 129 - RNA, 19 nt, Artificial
  YAP1
guuugaaucauagccuuga

SEQ ID NO: 130 - RNA, 19 nt, Artificial
  YAP1
caaaaguggguggcaauau

SEQ ID NO: 131 - RNA, 19 nt, Artificial
  YAP1
gaugaauuggaaaggagca

SEQ ID NO: 132 - RNA, 19 nt, Artificial
  YAP1
gccuuaguuuuggaaguaa

SEQ ID NO: 133 - RNA, 19 nt, Artificial
  YAP1
ggaagugacuuugcuacaa

SEQ ID NO: 134 - RNA, 19 nt, Artificial
  YAP1
gcucauauguuagguacuu

SEQ ID NO: 135 - RNA, 19 nt, Artificial
  YAP1
cuaguuuguaguucucauu

SEQ ID NO: 136 - RNA, 19 nt, Artificial
  YAP1
gcugccauuaaaggcagcu

SEQ ID NO: 137 - RNA, 19 nt, Artificial
  YAP1
ggcaugagacaauuuccau

SEQ ID NO: 138 - RNA, 19 nt, Artificial
  YAP1
ccuugauguggucucuugu

SEQ ID NO: 139 - RNA, 19 nt, Artificial
  YAP1
ccugcguagccaguuacca

SEQ ID NO: 140 - RNA, 19 nt, Artificial
  YAP1
gacucaaaauccagugucu

SEQ ID NO: 141 - RNA, 19 nt, Artificial
  YAP1
caagucugcaggaagcuuu

SEQ ID NO: 142 - RNA, 19 nt, Artificial
  YAP1
ggaagugagccuguuugga

SEQ ID NO: 143 - RNA, 19 nt, Artificial
  YAP1
gcuuuauagugguuuaccu

SEQ ID NO: 144 - RNA, 19 nt, Artificial
  YAP1
gcaugcucauauguuaggu

SEQ ID NO: 145 - RNA, 19 nt, Artificial
  YAP1
gcaccuaucacucucgaga

SEQ ID NO: 146 - RNA, 19 nt, Artificial
  YAP1
ggacuaagcaugagcagcu

SEQ ID NO: 147 - RNA, 19 nt, Artificial
  YAP1
gaguuugaaucauagccuu

SEQ ID NO: 148 - RNA, 19 nt, Artificial
  YAP1
gguggauuuuauccucgca

SEQ ID NO: 149 - RNA, 19 nt, Artificial
  YAP1
cauaagccaguugcaguuu

SEQ ID NO: 150 - RNA, 19 nt, Artificial
  YAP1
gugucuacaggaguaauaa

SEQ ID NO: 151 - RNA, 19 nt, Artificial
  YAP1
ucaugucacagcauuuagu

SEQ ID NO: 152 - RNA, 19 nt, Artificial
  YAP1
uguccuuguuccuaaugua

SEQ ID NO: 153 - RNA, 19 nt, Artificial
  YAP1
ucagucagggcuucuuaga

SEQ ID NO: 154 - RNA, 19 nt, Artificial
  YAP1
ucacucucgagaugagagu

SEQ ID NO: 155 - RNA, 19 nt, Artificial
  YAP1
ugaaggaucuaaggagaca

SEQ ID NO: 156 - RNA, 19 nt, Artificial
  YAP1
gaguaauaaugguuuccaa

SEQ ID NO: 157 - RNA, 19 nt, Artificial
  YAP1
uauuuuggcccuucaauuu

SEQ ID NO: 158 - RNA, 19 nt, Artificial
  YAP1
gagauggcaaagacaucuu

SEQ ID NO: 159 - RNA, 19 nt, Artificial
  YAP1
cagcagaauaugaugaacu

SEQ ID NO: 160 - RNA, 19 nt, Artificial
  YAP1
caccaagcuagauaaagaa

SEQ ID NO: 161 - RNA, 19 nt, Artificial
  YAP1
gcaccggaaauuuccauaa

SEQ ID NO: 162 - RNA, 19 nt, Artificial
  YAP1
ccaguggaaaaacaugauu

SEQ ID NO: 163 - RNA, 19 nt, Artificial
  YAP1
gauuaucugcucucucuuu

SEQ ID NO: 164 - RNA, 19 nt, Artificial
  YAP1
guccuuguuccuaauguaa

SEQ ID NO: 165 - RNA, 19 nt, Artificial
  YAP1
acagcauguucgagcucau

SEQ ID NO: 166 - RNA, 19 nt, Artificial
  YAP1
cuagaauaagcccuuauuu

SEQ ID NO: 167 - RNA, 19 nt, Artificial
  YAP1
gucucaggaauugagaaca

SEQ ID NO: 168 - RNA, 19 nt, Artificial
  YAP1
cuaaaucugugaaggaucu

SEQ ID NO: 169 - RNA, 19 nt, Artificial
  YAP1
gaacaaacguccagcaaga

SEQ ID NO: 170 - RNA, 19 nt, Artificial
  YAP1
guguucuagaaagagcuau

SEQ ID NO: 171 - RNA, 19 nt, Artificial
  YAP1
cauaaugugcaugacagaa

SEQ ID NO: 172 - RNA, 19 nt, Artificial
  YAP1
caccuaaguacacccacaa

SEQ ID NO: 173 - RNA, 19 nt, Artificial
  YAP1
gauguaagagcaugcucau

SEQ ID NO: 174 - RNA, 19 nt, Artificial
  YAP1
ggauggugggacucaaaau

SEQ ID NO: 175 - RNA, 19 nt, Artificial
  YAP1
gggcauacgguagauauua

SEQ ID NO: 176 - RNA, 19 nt, Artificial
  YAP1
cuagcaccucuguguuuua

SEQ ID NO: 177 - RNA, 19 nt, Artificial
  YAP1
ggaaggagugccuauaauu

SEQ ID NO: 178 - RNA, 19 nt, Artificial
  YAP1
gaaggagugccuauaauuu

SEQ ID NO: 179 - RNA, 19 nt, Artificial
  YAP1
ucaccuaaguacacccaca

SEQ ID NO: 180 - RNA, 19 nt, Artificial
  YAP1
ugagauaccugaugaugua

SEQ ID NO: 181 - RNA, 19 nt, Artificial
  YAP1
ucagggcuucuuagaucua

SEQ ID NO: 182 - RNA, 19 nt, Artificial
  YAP1
agauggaguuuuagaguag

SEQ ID NO: 183 - RNA, 19 nt, Artificial
  YAP1
cgacagucuucuuuugaga

SEQ ID NO: 184 - RNA, 19 nt, Artificial
  YAP1
gaauugagaacaaugacga

SEQ ID NO: 185 - RNA, 19 nt, Artificial
  YAP1
cucuguguuuuaagggucu

SEQ ID NO: 186 - RNA, 19 nt, Artificial
  YAP1
cuagaaugcaaaauugggu

SEQ ID NO: 187 - RNA, 19 nt, Artificial
  YAP1
guggauuuuauccucgcaa

SEQ ID NO: 188 - RNA, 19 nt, Artificial
  YAP1
ccuacuucuaugcugaaaa

SEQ ID NO: 189 - RNA, 19 nt, Artificial
  YAP1
ggaacaaaacgagcaugaa

SEQ ID NO: 190 - RNA, 19 nt, Artificial
  YAP1
gcaaucacuguguuguaua

SEQ ID NO: 191 - RNA, 19 nt, Artificial
  YAP1
ccuaaguacacccacaaaa

SEQ ID NO: 192 - RNA, 19 nt, Artificial
  YAP1
ggcuucuuagaucuacuua

SEQ ID NO: 193 - RNA, 19 nt, Artificial
  YAP1
accguuucccagacuaccu

SEQ ID NO: 194 - RNA, 19 nt, Artificial
  YAP1
uggaaugaacauagaagga

SEQ ID NO: 195 - RNA, 19 nt, Artificial
  YAP1
uugcucuuccuuguccauu

SEQ ID NO: 196 - RNA, 19 nt, Artificial
  YAP1
ucuuacgaugcccucugua

SEQ ID NO: 197 - RNA, 19 nt, Artificial
  YAP1
cuaugaaguaauaguuggu

SEQ ID NO: 198 - RNA, 19 nt, Artificial
  YAP1
caguuuucaggcuaauaca

SEQ ID NO: 199 - RNA, 19 nt, Artificial
  YAP1
cucagcuugggaagauaga

SEQ ID NO: 200 - RNA, 19 nt, Artificial
  YAP1
cagucagggcuucuuagau

SEQ ID NO: 201 - RNA, 19 nt, Artificial
  CHD1L
uucuuacugcggcuucaua

SEQ ID NO: 202 - RNA, 19 nt, Artificial
  CHD1L
cuggauaagcuacuagcau

SEQ ID NO: 203 - RNA, 19 nt, Artificial
  CHD1L
ggagccuuuugaaguugga

SEQ ID NO: 204 - RNA, 19 nt, Artificial
  CHD1L
cugcugcauaagaccuugu

SEQ ID NO: 205 - RNA, 19 nt, Artificial
  CHD1L
cugagucagcaagugaacu

SEQ ID NO: 206 - RNA, 19 nt, Artificial
  CHD1L
gagcuucccaagaagacag

SEQ ID NO: 207 - RNA, 19 nt, Artificial
  CHD1L
cguuccaauguccugucug

SEQ ID NO: 208 - RNA, 19 nt, Artificial
  CHD1L
ccuccucaagacagcuggu

SEQ ID NO: 209 - RNA, 19 nt, Artificial
  CHD1L
ggugggaauccaacaauua

SEQ ID NO: 210 - RNA, 19 nt, Artificial
  CHD1L
gcaucccaacuuacauaua

SEQ ID NO: 211 - RNA, 19 nt, Artificial
  CHD1L
cucaucgcauuggccaaaa

SEQ ID NO: 212 - RNA, 19 nt, Artificial
  CHD1L
gaagaaagcaaguguucau

SEQ ID NO: 213 - RNA, 19 nt, Artificial
  CHD1L
aguguucaucuuccacgua

SEQ ID NO: 214 - RNA, 19 nt, Artificial
  CHD1L
gucacguuuucaugugcua

SEQ ID NO: 215 - RNA, 19 nt, Artificial
  CHD1L
gaguguucuuguuguggau

SEQ ID NO: 216 - RNA, 19 nt, Artificial
  CHD1L
gcucagcaucgugaucguu

SEQ ID NO: 217 - RNA, 19 nt, Artificial
  CHD1L
cuuggccauuaagaacuuu

SEQ ID NO: 218 - RNA, 19 nt, Artificial
  CHD1L
gaugaagcucacagguuga

SEQ ID NO: 219 - RNA, 19 nt, Artificial
  CHD1L
ccuugucagaguucucagu

SEQ ID NO: 220 - RNA, 19 nt, Artificial
  CHD1L
cugaaacaggagucacguu

SEQ ID NO: 221 - RNA, 19 nt, Artificial
  CHD1L
cacguuuucaugugcuacu

SEQ ID NO: 222 - RNA, 19 nt, Artificial
  CHD1L
cagcaagugaacugcacaa

SEQ ID NO: 223 - RNA, 19 nt, Artificial
  CHD1L
gugauauaccauggcaugu

SEQ ID NO: 224 - RNA, 19 nt, Artificial
  CHD1L
cuuuggacagcagcccauu

SEQ ID NO: 225 - RNA, 19 nt, Artificial
  CHD1L
gagguacugcaauagagua

SEQ ID NO: 226 - RNA, 19 nt, Artificial
  CHD1L
cucagaaugacuugcaagc

SEQ ID NO: 227 - RNA, 19 nt, Artificial
  CHD1L
caagaccugaaacaggagu

SEQ ID NO: 228 - RNA, 19 nt, Artificial
  CHD1L
ccucaaguacguuaguggu

SEQ ID NO: 229 - RNA, 19 nt, Artificial
  CHD1L
cuuugucccuugucuguuu

SEQ ID NO: 230 - RNA, 19 nt, Artificial
  CHD1L
agaaggaggccauuuuacu

SEQ ID NO: 231 - RNA, 19 nt, Artificial
  CHD1L
agaaagcaaguguucaucu

SEQ ID NO: 232 - RNA, 19 nt, Artificial
  CHD1L
gggcaagauuuguuggccu

SEQ ID NO: 233 - RNA, 19 nt, Artificial
  CHD1L
gaaaaugagacggcaaaga

SEQ ID NO: 234 - RNA, 19 nt, Artificial
  CHD1L
ggagcaccauggaugaaau

SEQ ID NO: 235 - RNA, 19 nt, Artificial
  CHD1L
ccuggugggaauccaacaa

SEQ ID NO: 236 - RNA, 19 nt, Artificial
  CHD1L
uugaaaaugagacggcaaa

SEQ ID NO: 237 - RNA, 19 nt, Artificial
  CHD1L
ugauuggucgagacacugu

SEQ ID NO: 238 - RNA, 19 nt, Artificial
  CHD1L
aguugagugagauacucaa

SEQ ID NO: 239 - RNA, 19 nt, Artificial
  CHD1L
gugaacugcacaaacucuu

SEQ ID NO: 240 - RNA, 19 nt, Artificial
  CHD1L
cuccaagacuauauggauu

SEQ ID NO: 241 - RNA, 19 nt, Artificial
  CHD1L
cacuuggccauuaagaacu

SEQ ID NO: 242 - RNA, 19 nt, Artificial
  CHD1L
guguucaucuuccacguau

SEQ ID NO: 243 - RNA, 19 nt, Artificial
  CHD1L
ucagcaagugaacugcaca

SEQ ID NO: 244 - RNA, 19 nt, Artificial
  CHD1L
ggccgaucacuccgaaaua

SEQ ID NO: 245 - RNA, 19 nt, Artificial
  CHD1L
ccgaucacuccgaaauaaa

SEQ ID NO: 246 - RNA, 19 nt, Artificial
  CHD1L
ggcagaggugguuuauuua

SEQ ID NO: 247 - RNA, 19 nt, Artificial
  CHD1L
gggagguguccuuuuauuu

SEQ ID NO: 248 - RNA, 19 nt, Artificial
  CHD1L
gaccuugucagaguucuca

SEQ ID NO: 249 - RNA, 19 nt, Artificial
  CHD1L
gugguuuauuuacagcucu

SEQ ID NO: 250 - RNA, 19 nt, Artificial
  CHD1L
gcuugaaagaugcaucauu

SEQ ID NO: 251 - RNA, 19 nt, Artificial
  CHD1L
cccaaaugacccagauguu

SEQ ID NO: 252 - RNA, 19 nt, Artificial
  CHD1L
caagacccagaugcuacuu

SEQ ID NO: 253 - RNA, 19 nt, Artificial
  CHD1L
cagcuugcuaguugcauaa

SEQ ID NO: 254 - RNA, 19 nt, Artificial
  CHD1L
ugaacaacugguaaaccuu

SEQ ID NO: 255 - RNA, 19 nt, Artificial
  CHD1L
ucauugugcacugcguaga

SEQ ID NO: 256 - RNA, 19 nt, Artificial
  CHD1L
agguuuuaacugguauggu

SEQ ID NO: 257 - RNA, 19 nt, Artificial
  CHD1L
cugcaauagaguauuucaa

SEQ ID NO: 258 - RNA, 19 nt, Artificial
  CHD1L
gacuaccuaugagauuugc

SEQ ID NO: 259 - RNA, 19 nt, Artificial
  CHD1L
cgaggaugcucucauugug

SEQ ID NO: 260 - RNA, 19 nt, Artificial
  CHD1L
gauuuguuggccuugauug

SEQ ID NO: 261 - RNA, 19 nt, Artificial
  CHD1L
gcuucuuacugcggcuuca

SEQ ID NO: 262 - RNA, 19 nt, Artificial
  CHD1L
gcugacagggauucaccua

SEQ ID NO: 263 - RNA, 19 nt, Artificial
  CHD1L
cccugcugcauaagaccuu

SEQ ID NO: 264 - RNA, 19 nt, Artificial
  CHD1L
gcuucgaaaguguguggau

SEQ ID NO: 265 - RNA, 19 nt, Artificial
  CHD1L
gcugauuggucgagacacu

SEQ ID NO: 266 - RNA, 19 nt, Artificial
  CHD1L
gccaagagaaggagacuca

SEQ ID NO: 267 - RNA, 19 nt, Artificial
  CHD1L
cugcauaagaccuugucag

SEQ ID NO: 268 - RNA, 19 nt, Artificial
  CHD1L
gcuggauaagcuacuagca

SEQ ID NO: 269 - RNA, 19 nt, Artificial
  CHD1L
cuccgaaauaaaggcagug

SEQ ID NO: 270 - RNA, 19 nt, Artificial
  CHD1L
ccauuaagaacuuuggaca

SEQ ID NO: 271 - RNA, 19 nt, Artificial
  CHD1L
cagaaaacccuuuuggaga

SEQ ID NO: 272 - RNA, 19 nt, Artificial
  CHD1L
ggaguguucuuguugugga

SEQ ID NO: 273 - RNA, 19 nt, Artificial
  CHD1L
ggaugguucugugagagga

SEQ ID NO: 274 - RNA, 19 nt, Artificial
  CHD1L
cagcuggagggaguaaacu

SEQ ID NO: 275 - RNA, 19 nt, Artificial
  CHD1L
guugagugagauacucaaa

SEQ ID NO: 276 - RNA, 19 nt, Artificial
  CHD1L
gauuauucuaaagagccca

SEQ ID NO: 277 - RNA, 19 nt, Artificial
  CHD1L
cagaggugguuuauuuaca

SEQ ID NO: 278 - RNA, 19 nt, Artificial
  CHD1L
gcauuaagauggcagcccu

SEQ ID NO: 279 - RNA, 19 nt, Artificial
  CHD1L
ucaccaacaugaucauaga

SEQ ID NO: 280 - RNA, 19 nt, Artificial
  CHD1L
aguuggagaccaccugacu

SEQ ID NO: 281 - RNA, 19 nt, Artificial
  CHD1L
uggucgagacacuguggaa

SEQ ID NO: 282 - RNA, 19 nt, Artificial
  CHD1L
aacaauuaccaguccuucu

SEQ ID NO: 283 - RNA, 19 nt, Artificial
  CHD1L
ccuacgcucuuaccagcug

SEQ ID NO: 284 - RNA, 19 nt, Artificial
  CHD1L
cugacuaccuaugagauuu

SEQ ID NO: 285 - RNA, 19 nt, Artificial
  CHD1L
ggugggagauuuuauucaa

SEQ ID NO: 286 - RNA, 19 nt, Artificial
  CHD1L
gaccuagaugcauuugaaa

SEQ ID NO: 287 - RNA, 19 nt, Artificial
  CHD1L
caccaacaugaucauagaa

SEQ ID NO: 288 - RNA, 19 nt, Artificial
  CHD1L
ccuccaguugagugagaua

SEQ ID NO: 289 - RNA, 19 nt, Artificial
  CHD1L
cugguaaaccuucagaaaa

SEQ ID NO: 290 - RNA, 19 nt, Artificial
  CHD1L
gcucugcugagcuggauua

SEQ ID NO: 291 - RNA, 19 nt, Artificial
  CHD1L
ccgaggaugcucucauugu

SEQ ID NO: 292 - RNA, 19 nt, Artificial
  CHD1L
caauguccugucuggcauu

SEQ ID NO: 293 - RNA, 19 nt, Artificial
  CHD1L
auagaaggaggccauuuua

SEQ ID NO: 294 - RNA, 19 nt, Artificial
  CHD1L
acugcaauagaguauuuca

SEQ ID NO: 295 - RNA, 19 nt, Artificial
  CHD1L
caguugagugagauacuca

SEQ ID NO: 296 - RNA, 19 nt, Artificial
  CHD1L
ccuuaagaauuggcccagc

SEQ ID NO: 297 - RNA, 19 nt, Artificial
  CHD1L
ucccugcugcauaagaccu

SEQ ID NO: 298 - RNA, 19 nt, Artificial
  CHD1L
gcuugcuaguugcauaaua

SEQ ID NO: 299 - RNA, 19 nt, Artificial
  CHD1L
gcuggugccuuaagaauug

SEQ ID NO: 300 - RNA, 19 nt, Artificial
  CHD1L
aacuuuggacagcagccca

SEQ ID NO: 301 - RNA, 19 nt, Artificial
  siCONT
cuuacgcugaguacuucga

SEQ ID NO: 302 - DNA, 20 bp, Artificial
  GAPDH-F
ggtgaaggtcggagtcaacg

SEQ ID NO: 303 - DNA, 25 bp, Artificial
  GAPDH-R
accatgtagttgaggtcaatgaagg

SEQ ID NO: 304 - DNA, 20 bp, Artificial
  ZBTB7A-F
agtgcttctcctggccgttg

SEQ ID NO: 305 - DNA, 20 bp, Artificial
  ZBTB7A-R
cgaccacctgcacagacacc

SEQ ID NO: 306 - DNA, 20 bp, Artificial
  YAP1-F
gaaccgtttcccagactacc

SEQ ID NO: 307 - DNA, 20 bp, Artificial
  YAP1-R
gcatcagctcctctccttct

SEQ ID NO: 308 - DNA, 19 bp, Artificial
  CHD1L-F
ccgatcactccgaaataaa

SEQ ID NO: 309 - DNA, 20 bp, Artificial
  CHD1L-R
gcctcttccttttgcctctt

SEQ ID NO: 310 - RNA, 19 nt, Artificial
  ZBTB7A_Ref
gggcgucauggacuacuac

SEQ ID NO: 311 - RNA, 19 nt, Artificial
  YAP1_Ref
gacaucuucuggucagaga

SEQ ID NO: 312 - RNA, 21 nt, Artificial
  CHD1L_Ref
cguauuggacaugccacgaaa

(19) United States
(12) Patent Application Publication
(10) Pub. No.: US 2016/0145624 A1
(43) Pub. Date: May 26, 2016

(54) LIVER CANCER RELATED GENES-SPECIFIC siRNA, DOUBLE-STRANDED OLIGO RNA MOLECULES COMPRISING THE siRNA, AND COMPOSITION FOR PREVENTING OR TREATING CANCER COMPRISING THE SAME

(71) Applicants: BIONEER CORPORATION, Daedeok-gu, Daejeon (KR); SANOFI-AVENTIS KOREA CO., LTD., Seocho-gu, Seoul (KR)

(72) Inventors: Jeiwook Chae, Daejeon (KR); Pyoung Oh Yoon, Daejeon (KR); Han-na Kim, Jeollabuk-do (KR); Han Oh Park, Daejeon (KR); Boram Han, Daejeon (KR); Youngho Ko, Seoul (KR); Gi-Eun Choi, Gyeonggi-do (KR); Jae Eun Kim, Seoul (KR)

(21) Appl. No.: 14/903,043

(22) PCT Filed: Jul. 9, 2014
(86) PCT No.: PCT/KR14/06146

§ 371 Date: Jan. 5, 2016

(30) Foreign Application Priority Data
Jul. 9, 2013 (KR) 10-2013-0080580

(51) Int. Cl. C12N 15/113; A61K 31/713; A61K 47/48

(52) U.S. Cl. CPC C12N 15/1137; A61K 47/488; A61K 31/713; C12N 15/113; C12N 15/1135; C12N 2310/351; C12N 2310/31; C12N 2310/14; C12N 2320/30

(57) ABSTRACT

There is provided a liver cancer related specific siRNA and high efficiency double-stranded oligo RNA molecules containing the same. The double-stranded oligo RNA molecules have a structure in which hydrophilic and hydrophobic compounds are conjugated to both ends of the double-stranded oligo RNA molecules by a simple covalent bond or a linker-mediated covalent bond in order to be efficiently delivered into cells and may be converted into nanoparticles in an aqueous solution by hydrophobic interactions of the double-stranded oligo RNA molecules. The siRNA contained in the double-stranded oligo RNA molecules may be liver cancer related genes, particularly ZBTB7A, YAP1 or CHD1L specific siRNA.

In addition, the present invention relates to a method of preparing the double-stranded oligo RNA molecules, and a pharmaceutical composition for preventing or treating cancer, particularly, liver cancer, containing the double-stranded oligo RNA molecules.

### TECHNICAL FIELD

The present invention relates to a liver cancer related specific siRNA and high efficiency double-stranded oligo RNA molecules containing the same. The double-stranded oligo RNA molecules have a structure in which hydrophilic and hydrophobic compounds are conjugated to both ends of the double-stranded RNA molecules by a simple covalent bond or a linker-mediated covalent bond in order to be efficiently delivered into cells and may be converted into nanoparticles in an aqueous solution by hydrophobic interactions of the double-stranded oligo RNA molecules. The siRNA contained in the double-stranded oligo RNA molecules may be preferably liver cancer related genes, particularly ZBTB7A, YAP1 or CHD1L, specific siRNA.

In addition, the present invention relates to a method of preparing the double-stranded oligo RNA molecules, and a pharmaceutical composition for preventing or treating cancer, particularly, liver cancer, containing the double-stranded oligo RNA molecules.

### BACKGROUND ART

A technology of suppressing expression of genes is an important tool in developing a therapeutic agent for treating diseases and validating a target. Among these technologies, since roles of RNA interference (hereinafter, referred to as ‘RNAi’) was found, it was found that the RNAi acts on sequence-specific mRNA in various kinds of mammalian cells (Silence of the Transcripts: RNA Interference in Medicine, J. Mol. Med. 83: 764-773, 2005). When long-chain double-stranded RNA is delivered into cells, the delivered double stranded RNA is converted into a small interfering RNA (hereinafter, referred to as ‘siRNA’) processed into 21 to 23 base pairs (bp) by endonuclease called Dicer, wherein the siRNA is bound to a RNA-induced silencing complex (RISC), and then a guide (antisense) strand recognizes and degrades the target mRNA, such that the siRNA sequence-specifically inhibits expression of the target gene (Nucleic-Acid Therapeutics: Basic Principles and Recent Applications, Nature Reviews Drug Discovery 1: 503-514, 2002).

According to Bertrand et al., it was found that the siRNA has a more excellent effect of inhibiting expression of the mRNA in vivo and in vitro as compared to antisense oligonucleotide (ASO) on the same target genes (Comparison of Antisense Oligonucleotides and siRNAs in Cell Culture and in Vivo, Biochem. Biophys. Res. Commun, 296: 1000-1004, 2002). In addition, since action mechanism of the siRNA is that the siRNA is complementarily bound to the target mRNA to sequence-specifically control the expression of the target genes, the target to which the siRNA may be applied may be remarkably enlarged as compared to the existing antibody based drugs or small molecule drugs (Progress towards in Vivo Use of siRNAs, Molecular Therapy 13(4): 664-670, 2006).

In spite of excellent effects and various uses of the siRNA, in order to develop the siRNA as a therapeutic agent, the siRNA should be effectively delivered into a target cell by improving stability of the siRNA in vivo and cell delivery efficiency (Harnessing in vivo siRNA delivery for drug discovery and therapeutic development, Drug Discov. Today 11(1-2): 67-73, January 2006).

In order to solve the above-mentioned problem, research into a technology of modifying some nucleotides or a backbone of the siRNA for improving the stability in vivo so as to have resistance against nuclease or using a carrier such as a viral vector, liposome, nanoparticles, or the like, has been actively conducted.

In a delivery system using the viral vector such as adenovirus, retrovirus, or the like, transfection efficiency is high, but immunogenicity and oncogenicity are also high. On the other hand, a non-viral delivery system including nanoparticles has low cell delivery efficiency as compared to the viral delivery system but has advantages in that the non-viral delivery system may have high stability in vivo, be target-specific delivered, improve delivery efficiency through uptake or internalization of RNAi oligonucleotide contained therein into cell or tissues, or the like, and does not almost cause cytotoxicity and immune stimulation, such that currently, the non-viral delivery system has been evaluated as a potential delivery system as compared to the viral delivery system (Nonviral Delivery of Synthetic siRNA in vivo, J. Clin. Invest., 117(12): 3623-3632, Dec. 3, 2007).

Among the non-viral delivery systems, in a method of using nanocarrier, nanoparticles are formed by using various polymers such as liposome, a cationic polymer complex, and the like, and then iRNA is supported on these nanoparticles, that is, nanocarriers to thereby be delivered into the cell. In the method of using the nanocarrier, a polymeric nanoparticle, polymer micelle, lipoplex, and the like, are mainly used. Among them, the lipoplex is composed of cationic lipid and interacts with anionic lipid of endosome in the cell to destabilize the endosome, thereby serving to deliver the iRNA into the cell (Proc. Natl. Acad. Sci. 15; 93(21): 11493-8, 1996).

In addition, it was known that the efficiency of the siRNA in vivo may be increased by conjugating chemical compound, or the like, to an end site of a passenger (sense) strand of the siRNA to allow the siRNA to have improved pharmacokinetic features (Nature 11; 432(7014): 173-8, 2004). In this case, the stability of the siRNA may be changed according to the property of the chemical compound conjugated to the end of the sense (passenger) or antisense (guide) strand of the siRNA. For example, siRNA conjugated with a polymer compound such as polyethylene glycol (PEG) interacts with an anionic phosphoric acid group of the siRNA in a presence of cationic compound to form a complex, thereby obtaining a carrier comprising improved siRNA stability (J. Control Release 129(2): 107-16, 2008). Particularly, since micelles made of a polymer complex have significantly uniform distribution and a structure spontaneously formed while comprising significantly small sizes as compared to microsphere, nanoparticles, or the like, which is another system used as a drug delivery carrier, there are advantages in that quality of a product may be easily managed and reproducibility may be easily secured.

Further, in order to improve intracellular delivery efficiency of the siRNA, a technology for securing stability of the siRNA and implementing efficient cell membrane permeability using a siRNA conjugate obtained by conjugating a hydrophilic compound (for example, polyethylene glycol (PEG)), which is a biocompatible polymer, to the siRNA via a simple covalent bond or a linker-mediated covalent bond has been developed (Korean Patent Registration No. 883471). However, even though the siRNA is chemically modified and conjugated to polyethylene glycol (PEC), disadvantages such as low stability in vivo and difficulty in delivering the siRNA into a target organ still remains. In order to solve these disadvantages, double-stranded oligo RNA molecules in which hydrophilic and hydrophobic compounds are bound to oligonucleotide, particularly, double-stranded oligo RNA such as siRNA have been developed. The molecules form self-assembled nanoparticles (which is referred to as self-assembled micelle inhibitory RNA (SAMiRNA™)) by hydrophobic interaction of the hydrophobic compound (See Korean Patent Registration No. 1224828). A SAMiRNA™ technology has advantages in that homogenous nanoparticles comprising significantly small sizes as compared to the existing delivery technologies may be obtained.

Meanwhile, one in four Koreans die due to cancer (first cause of death), and in accordance with the development of a diagnostic method and a date collecting method, aging population, environmental changes, and the like, the number of patients die due to cancer is significantly increased every year. In addition, generation of cancer and death due to cancer has also been increased in the world, such that a technology of preventing, diagnosing, and treating cancer is a common and urgent task for people (Bio-Technology (BT) Trends Report, Current Development Trend of New Drug for Major Diseases, Biotechnology Policy Research Center, 2007, Edition No. 72).

Cancer is one of the diseases resulting in death to the largest number of people around the world, and the development of an innovative cancer therapeutic agent may decrease medical expenses consumed at the time of treating cancer and create high added-value. Therapy of cancer is divided into surgery, radiation therapy, chemotherapy, and biological therapy. Among them, chemotherapy is a therapeutic method of suppressing proliferation of cancer cells or killing the cancer cells using a small molecule drug. Since much of the toxicities expressed by an anticancer drug are shown in normal cells, the anticancer drug has toxicity at some degree. In addition, the anticancer drug has resistance in that the drug has an anticancer effect but loses the anticancer effect after the drug is used for a constant period. Therefore, development of an anticancer drug capable of selectively acting on cancer cells and not generating resistance has been urgently demanded (Current Status of Conquering Cancer. BioWave 6 (19), 2004). Recently, development of a new anticancer drug target molecular features of cancer by securing molecular genetic information on cancer has been conducted, and it was reported that drug resistance is not generated in anticancer drugs targeting a specific molecular target. Therefore, a therapeutic agent comprising excellent effects and reducing adverse effects as compared to the existing anticancer drug may be developed by developing a gene therapeutic agent targeting the specific molecular target which only cancer cells have.

After it was known that expression of genes may be specifically and efficiently inhibited using a RNA interference phenomenon, research into siRNAs targeting various genes has been conducted as a therapeutic drug for cancer. Examples of these genes may include oncogene, an anti-apoptotic molecule, telomerase, growth factor receptor gene, signaling molecule, and the like, the research is mainly conducted toward inhibiting expression of genes required for survival of cancer cells or inducing apotosis (RNA Interference in Cancer, Biomolecular Engineering 23: 17-34, 2006).

ZBTB7A (zinc finger and BTB domain containing 7A) is proto-oncogene belonging to POK (POZ and Krüppel) known as transcription inhibitor. ZBTB7A is known to specifically inhibit transcription of ARF (alternate reading frame of the INK4a/ARF locus (CDKN2A)) which is tumor suppressor gene, and inactivate p53 indirectly (Won-II Choi et al. (2009) Proto-oncogene FBI-1 Represses Transcription of p21CIP1 by Inhibition of Transcription Activation by p53 and Sp1. THE JOURNAL OF BIOLOGICAL CHEMISTRY 284(19):12633-12644).

In other words, as a master controller of ARF-Hdm2-p53-p21 pathway, the ZBTB7A influences p21, cell cycle arrest factor, by inhibiting upstream regulator in transcription or expression (translational) level. Thus the ZBTB7A accelerates cell proliferation, increases significantly the number of cells in S phase. In addition, the ZBTB7A is abnormally overexpressed solid tumors, including liver cancer (FBI-1 promotes cell proliferation and enhances resistance to chemotherapy of hepatocellular carcinoma in vitro and in vivo. Cancer [2012, 118(1):134-146).

YAP1 (Yes-associated protein 1) was known to be binding to SH3 domain of Src protein tyrosine-kinase (Sudol M (1994) Yes-associated protein (YAP65) is a proline-rich phosphoprotein that binds to the SH3 domain of the Yes proto-oncogene product. Oncogene 9: 2145-2152). The YAP1 is potential oncogene overexpressed in various human cancers, and one of main effector of Hippo tumor inhibition pathway (Pan D (2010), The hippo signaling pathway in development and cancer. Dev Cell 19: 491-505). The YAP1 acts as co-activator with TEAD transcription factor in transcription process, and increases expression of genes which facilitate cell proliferation and inhibit apoptosis (Zhao B, Kim J, Ye X, Lai Z C, Guan K L (2009) Both TEAD-binding and WW domains are required for the growth stimulation and oncogenic transformation activity of yes-associated protein. Cancer Res 69: 1089-98). The Hippo tumor suppression pathway, downstream effector of YAP is known to be associated with CREB (cAMP response element-binding protein) in liver cancer formation (Mutual interaction between YAP and CREB promotes tumorigenesis in liver cancer. Hepatology. 2013 Mar. 26).

CHD1L (1-like Chromodomain-helicase-DNA-binding protein) is known to be related to the chromatin remodeling and DNA relaxation process required for DNA replication, repair and transcription (Poly(ADP-ribose)-dependent regulation of DNA repair by the chromatin remodeling enzyme ALC1. Science. 2009 Sep. 4; 325(5945):1240-3). The CHD1L, also known as ALC1 (amplified in liver cancer 1) is frequently amplified and overexpressed in liver cancer (human hepatocelluar carcinoma, HCC). In addition, overexpression of CHD1L facilitates transition of G1/S phase in cell cycle, cell proliferation by reducing p53 expression, and inhibits apoptosis (Isolation and characterization of a novel oncogene, amplified in liver cancer 1, within a commonly amplified region at 1q21 in hepatocellular carcinoma. Hepatology. 2008 February; 47(2): 503-10).

As described above, possibilities of ZBTB7A, YAP1 and CHD1L as targets for anti-cancer drug are known, but development of a siRNA therapeutic agent for ZBTB7A, YAP1 or CHD1L and a technology of delivering the siRNA therapeutic agent is still insignificant. Therefore, a need for the siRNA therapeutic agent capable of specifically and efficiently inhibiting expression of ZBTB7A, YAP1 or CHD1L and the technology of delivering the siRNA therapeutic agent is significant in the market.

### DISCLOSURE

### Technical Problem

An object of the present invention is to provide a new siRNA capable of specifically and highly efficiently inhibiting expression of ZBTB7A, YAP1 or CHD1L, double-stranded oligo RNA molecules containing the same, and a method of preparing the double-stranded oligo RNA molecules.

Another object of the present invention is to provide a pharmaceutical composition for preventing or treating cancer, particularly, liver cancer, containing ZBTB7A, YAP1 or CHD1L specific-siRNA or double-stranded oligo RNA molecules containing the ZBTB7A, YAP1 or CHD1L specific siRNA as an active ingredient.

Still another object of the present invention is to provide a method of preventing or treating cancer using the ZBTB7A, YAP1 or CHD1L specific-siRNA or the double-stranded oligo RNA molecules containing the ZBTB7A, YAP1 or CHD1L specific siRNA.

### Technical Solution

According to an aspect of the present invention, there is provided ZBTB7A, YAP1 or CHD1L specific siRNA, which is liver cancer related gene, comprising a first oligonucleotide, which is a sense strand comprising any one sequence selected from SEQ ID NOs. 1 to 300 and a second oligonucleotide, which is an antisense strand complementary thereto.

The term “ZBTB7A specific siRNA(s)”, “YAP1 specific siRNA(s)” or “CHD1L specific siRNA(s)” of the present invention means an siRNA(s) which is specific for gene encoding ZBTB7A, YAP1 or CHD1L protein.

In addition, as long as the siRNAs retain the specificity to ZBTB7A, YAP1 or CHD1L, the siRNAs of the present invention also comprise sense or antisense strand having one or more nucleotide deletion, insertion or substitution in sense strand of SEQ ID NOs:1 to 300 or antisense strand complementary to the SEQ ID NOs:1 to 300.

The SEQ ID NOs. 1 to 100 indicate sequences of the sense strand of the ZBTB7A specific siRNA, the SEQ ID NOs. 101 to 200 indicate sequences of the sense strand of the YAP1 specific siRNA, and the SEQ ID NOs. 201 to 300 indicate sequences of the sense strand of the CHD1L specific siRNA.

Preferably, the siRNA according to the present invention may have a sense strand of the ZBTB7A specific siRNA comprising a sequence of the SEQ ID NO. 1, 2, 3, 4, 5, 6, 7 or 8, a sense strand of the YAP1 specific siRNA comprising a sequence of the SEQ ID NO. 103, 107, 108, 112, 116, 117, 118, 121 or 122, or a sense strand of the CHD1L specific siRNA comprising a sequence of the SEQ ID NO. 201, 202, 203 or 204,

more preferably, the siRNA according to the present invention may have a sense strand of the ZBTB7A specific siRNA comprising a sequence of the SEQ ID NO. 1, 2 or 4, a sense strand of the YAP1 specific siRNA comprising a sequence of the SEQ ID NO. 116, 118 or 121, or a sense strand of the CHD1L specific siRNA comprising a sequence of the SEQ ID NO. 202, 203 or 204,

most preferably the siRNA according to the present invention may have a sense strand of the ZBTB7A specific siRNA comprising a sequence of the SEQ ID NO. 4, a sense strand of the YAP1 specific siRNA comprising a sequence of the SEQ ID NO. 118, or a sense strand of the CHD1L specific siRNA comprising a sequence of the SEQ ID NO. 204.

The sense strand or antisense strand of the siRNA according to the present invention may be composed of 19 to 31 nucleotides.

Since the ZBTB7A, YAP1 or CHD1L specific siRNA provided in the present invention has a base sequence designed so as to be complementarily bound to mRNA encoding a gene corresponding thereto, the ZBTB7A, YAP1 or CHD1L specific siRNA may effectively suppress the expression of the corresponding gene. In addition, the ZBTB7A, YAP1 or CHD1L specific siRNA may include an overhang, which is a structure comprising one or at least two unpaired nucleotides at a 3′-end of the siRNA,

and in order to improve the stability of the siRNA in vivo, the ZBTB7A, YAP1 or CHD1L specific siRNA may include various modifications for imparting resistance against nuclease and decreasing non-specific immune reactions. Describing modification of the first or second oligonucleotide configuring the siRNA, at least one modification selected from modification by substitution of —OH group with —CH₃ (methyl), —OCH₃ (methoxy), —NH₂, —F (fluorine), —O-2-methoxyethyl, —O-propyl, —O-2-methylthioethyl, —O-3-aminopropyl, —O-3-dimethylaminopropyl, —O—N-methylacetamido, or —O-dimethylamidooxyethyl at a 2′-carbon site of a sugar structure in at least one nucleotide; modification by substitution of oxygen in the sugar structure in the nucleotide with sulfur; modification of a nucleotide bond into a phosphorothioate bond, a boranophosphate bond, or a methyl phosphonate bond may be combined to thereby be used, and modification into a peptide nucleic acid (PNA) type, a locked nucleic acid (LNA) type, or a unlocked nucleic acid (UNA) type may be used (Ann. Rev. Med. 55, 61-65 2004; U.S. Pat. No. 5,660,985; U.S. Pat. No. 5,958,691; U.S. Pat. No. 6,531,584; U.S. Pat. No. 5,808,023; U.S. Pat. No. 6,326,358; U.S. Pat. No. 6,175,001; Bioorg. Med. Chem. Lett. 14:1139-1143, 2003; RNA, 9:1034-1048, 2003; Nucleic Acid Res. 31:589-595, 2003; Nucleic Acids Research, 38(17) 5761-5773, 2010; Nucleic Acids Research, 39(5) 1823-1832, 2011).

The ZBTB7A, YAP1 or CHD1L specific siRNA provided in the present invention may significantly inhibit expression of corresponding proteins in addition to inhibiting expression the corresponding gene. Further, since it was known that the siRNA may improve sensitivity of radiation therapy or chemotherapy, which is a therapeutic method typically combined with a cancer-specific RNAi used to treat cancer (The Potential RNAi-based Combination Therapeutics. Arch. Pharm. Res. 34(1): 1-2, 201), the ZBTB7A, YAP1 or CHD1L specific siRNA according to the present invention may be used together with the existing radiation therapy or chemotherapy.

Further, in the case in which the ZBTB7A, YAP1 and CHD1L specific siRNA according to the present invention are simultaneously used, expression of the corresponding genes is simultaneously inhibited, such that growth of cancer cells may be remarkably inhibited.

According to another aspect of the present invention, there is provided a conjugate in which hydrophilic and hydrophobic compounds are conjugated to both ends of the siRNA in order to efficiently deliver the liver cancer related genes, particularly ZBTB7A, YAP1 or CHD1L specific siRNA into the body and improve stability.

In the case in which the hydrophilic and hydrophobic compounds are bound to the siRNA as described above, self assembled nanoparticles are formed by the hydrophobic interaction of the hydrophobic compound (See Korean Patent Registration No. 1224828). This conjugate has significantly excellent delivery efficiency into the body and excellent stability in vivo, and uniformity of particles sizes is excellent, such that quality control (QC) may be easy. Therefore, this conjugate may have advantages in that a preparing process as a drug is simple.

As a specific example, the double-stranded oligo RNA molecules containing ZBTB7A, YAP1 or CHD1L specific siRNA according to the present invention may preferably have a structure of the following Structural Formula (1).

A-X—R—Y—B Structural Formula (1)

In Structural Formula (1), A is a hydrophilic compound, B is a hydrophobic compound, X and Y each are independently a simple covalent bond or linker-mediated covalent bond, and R is ZBTB7A, YAP1 or CHD1L specific siRNA.

More preferably, the double-stranded oligo RNA molecules containing ZBTB7A, YAP1 or CHD1L specific siRNA according to the present invention may have a structure of the following Structural Formula (2).

A-X—S—Y—B

AS Structural Formula (2)

In Structural Formula (2), A, B, X, and Y have the same definitions as those in Structural Formula (1), respectively, S is a sense strand of the ZBTB7A, YAP1 or CHD1L specific siRNA, and AS is an antisense strand of the ZBTB7A, YAP1 or CHD1L specific siRNA.

As long as the siRNAs retain the specificity to ZBTB7A, YAP1 or CHD1L, the ZBTB7A, YAP1 or CHD1L specific siRNAs of the present invention also comprise antisense strand which is partially complementary (mismatch) to the ZBTB7A, YAP1 or CHD1L mRNA, as well as antisense strand perfectly complementary (perfect match) to the ZBTB7A, YAP1 or CHD1L mRNA.

The antisense or sense strand of the siRNA of the present invention may have at least 70%, preferably 80%, more preferably 90%, and most preferably 95% of sequence homology or complementarity to the ZBTB7A, YAP1 or CHD1L mRNA sequence.

The siRNA may be a double stranded duplex or single stranded polynucleotide including, but not limited to, antisense oligonucleotide or miRNA.

More preferably, the double-stranded oligo RNA molecules containing ZBTB7A, YAP1 or CHD1L specific siRNA according to the present invention may have a structure of the following Structural Formula (3).

A-X-5′S3′-Y—B

AS Structural Formula (3)

It will be apparent to those skilled in the art to which the present invention pertains that in Structural Formulas (1) to (3), one to three phosphate groups may be bound to a 5′-end of the antisense strand of the double-stranded oligo RNA molecules containing ZBTB7A, YAP1 or CHD1L specific siRNA and siRNA may be used instead of the siRNA.

The hydrophilic compound in Structural Formulas (1) to (3) may be preferably a cationic or non-ionic polymer compound comprising a molecular weight of 200 to 10,000, more preferably a non-ionic polymer compound comprising a molecular weight of 1,000 to 2,000. For example, as a hydrophilic polymer compound, a non-ionic hydrophilic polymer compound such as polyethylene glycol, polyvinyl pyrrolidone, polyoxazoline, and the like, may be preferably used, but the present invention is not limited thereto.

The hydrophobic compound B in Structural Formulas (1) to (3) may serve to form nanoparticles made of oligonucleotide molecules of Structural Formula (1) through the hydrophobic interaction. Preferably, the hydrophobic compound may have a molecular weight of 250 to 1,000, and a steroid derivative, a glyceride derivative, glycerol ether, polypropylene glycol, saturated or unsaturated C₁₂-C₅₀ hydrocarbon, diacyl phosphatidylcholine, fatty acid, phospholipid, lipopolyamine, or the like, may be used, but the present invention is not limited thereto. It may be apparent to those skilled in the art to which the present invention pertains that any hydrophobic compound may be used as long as the compound may satisfy the object of the present invention.

The steroid derivative may be selected from a group consisting of cholesterol, cholestanol, cholic acid, cholesteryl formate, cholestanyl formate, and cholesteryl amine, and the glyceride derivative may be selected from mono-, di-, and tri-glycerides, and the like. In this case, fatty acid of the glyceride may be preferably unsaturated or saturated C₁₂-C₅₀ fatty acid.

Particularly, among the hydrophobic compounds, the saturated or unsaturated hydrocarbon or cholesterol may be preferable in that they may be easily bound in a process of synthesizing the oligonucleotide molecules according to the present invention.

The hydrophobic compound may be bound to a distal end opposite to the hydrophilic compound and may be bound to any site of the sense or antisense strand of the siRNA.

The hydrophilic or hydrophobic compound in Structural Formulas (1) to (3) and the ZBTB7A, YAP1 or CHD1L specific siRNA according to the present invention may be bound to each other by a simple covalent bond or a linker-mediated covalent bond (X or Y). The linker mediating the covalent bond is covalently bound to the hydrophilic or hydrophobic compound at the end of the ZBTB7A, YAP1 or CHD1L specific siRNA, and as long as the linker may provide a degradable bond in a specific environment, as needed, the linker is not particularly limited. Therefore, as the linker, any compound bound in order to activate the ZBTB7A, YAP1 or CHD1L specific siRNA and/or the hydrophilic (or hydrophobic) compound in the process of preparing the double-stranded oligo RNA molecules according to the present invention may be used. The covalent bond may be any one of a non-degradable bond or a degradable bond. In this case, examples of the non-degradable bond may include an amide bond and a phosphate bond, and examples of the degradable bond may include a disulfide bond, an acid-degradable bond, an ester bond, an anhydride bond, a biodegradable bond, an enzyme-degradable bond, and the like, but the non-degradable or the degradable bond are not limited thereto.

In addition, as the ZBTB7A, YAP1 or CHD1L specific siRNA represented by R in Structural Formulas (1) to (3), any siRNA may be used without limitations as long as the siRNA may be specifically bound to ZBTB7A, YAP1 or CHD1L. Preferably, in the present invention, the ZBTB7A, YAP1 or CHD1L specific siRNA is composed of the sense strand comprising any one sequence selected from the SEQ ID NOs. 1 to 300 and the antisense strand comprising a sequence complementary thereto.

The siRNA according to the present invention may have preferably a sense strand of the ZBTB7A specific siRNA comprising a sequence of the SEQ ID NO. 1, 2, 3, 4, 5, 6, 7 or 8, a sense strand of the YAP1 specific siRNA comprising a sequence of the SEQ ID NO. 103, 107, 108, 112, 116, 117, 118, 121 or 122, or a sense strand of the CHD1L specific siRNA comprising a sequence of the SEQ ID NO. 201, 202, 203 or 204,

more preferably, the siRNA according to the present invention may have a sense strand of the ZBTB7A specific siRNA comprising a sequence of the SEQ ID NO. 1, 2 or 4, a sense strand of the YAP1 specific siRNA comprising a sequence of the SEQ ID NO. 116, 118 or 121, or a sense strand of the CHD1L specific siRNA comprising a sequence of the SEQ ID NO. 202, 203 or 204,

most preferably the siRNA according to the present invention may have a sense strand of the ZBTB7A specific siRNA comprising a sequence of the SEQ ID NO. 4, a sense strand of the YAP1 specific siRNA comprising a sequence of the SEQ ID NO. 118, or a sense strand of the CHD1L specific siRNA comprising a sequence of the SEQ ID NO. 204.

Meanwhile, tumor tissue is significantly rigid and has diffusion-limitation as compared with normal tissue. Since this diffusion-limitation has a negative influence on movement of nutrients required for tumor growth, oxygen, waste materials such as carbon dioxide, the tumor tissue overcomes this diffusion-limitation by forming a blood vessel therearound through angiogenesis. The blood vessel generated through the angiogenesis in the tumor tissue may be a leaky and defective blood vessel comprising a leak of 100 nm to 2 um according to a kind of cancer. Therefore, the nanoparticles may easily pass through capillary endothelium of the cancer tissue comprising the leaky and defective structure as compared to organized capillary vessels of the normal tissue, such that the nanoparticles may easily approach the tumor interstitium during a circulation process in a blood vessels, and lymphatic drainage does not exist in the tumor tissue, such that drugs may be accumulated, which is called an ‘enhanced permeation and retention (EPR) effect’. Nanoparticles are tumor tissue-specifically delivered by this effect, which is referred to as ‘passive targeting’ (Nanoparticles for Drug Delivery in Cancer Treatment, Urol. Oncol., 26(1): 57-64, January-February, 2008). Active targeting means that a targeting moiety is bound to nanoparticles, and it was reported that the targeting moiety promotes preferential accumulation of the nanoparticles in the target tissue or improves internalization of the nanoparticles into the target cells (Does a Targeting Ligand Influence Nanoparticle Tumor Localization or Uptake Trends, Biotechnol. 26(10): 552-8m October, 2008, Epub. Aug. 21, 2008). In the active targeting, a target cell-specific material or a material, that is, the target moiety, capable of binding to over-expressed carbohydrate, receptor, or antigen is used (Nanotechnology in Cancer Therapeutics: Bioconjugated Nanoparticles for Drug Delivery, Mol. Cancer Ther., 5(8): 1909-1917, 2006).

Therefore, in the case in which the targeting moiety is provided in the double-stranded oligo RNA molecules containing ZBTB7A, YAP1 or CHD1L specific siRNA according to the present invention and the nanoparticles formed therefrom, delivery of the siRNA into the target cell may be efficiently promoted, such that the siRNA may be delivered into the target cell even at a relatively low concentration to thereby exhibit a high target gene expression regulatory function and prevent the ZBTB7A, YAP1 or CHD1L specific siRNA from being non-specifically delivered to other organs or cells.

Accordingly, the present invention provides double-stranded oligo RNA molecules in which a ligand L, particularly, a ligand specifically bound to a receptor promoting the internalization into the target cell through receptor-mediated endocytosis (RME) is additionally bound to the molecules represented by Structural Formulas (1) to (3), and a form in which the ligand is bound to the double-stranded RNA molecules represented by Structural Formula (1) has a structure of the following Structural Formula (4).

(Lᵢ-Zⱼ)-A-X—R—Y—B Structural Formula (4)

In Structural Formula (4), A, B, X, and Y have the same definitions as those in Structural Formulas (1) to (3), respectively, L is a ligand specifically bound to the receptor promoting the internalization into the target cell through receptor-mediated endocytosis (RME), and i and j each are independently 0 or 1.

Preferably, the ligand in Structural Formula (5) may be selected from a target receptor-specific antibody, aptamer, and peptide that have a receptor-mediated endocytic (REM) effect of target cell specifically promoting internalization; and chemicals, for example, folate (generally folate and folic acid are compatible with each other, and folate in the present invention means natural folate or active folate in the body), hexoamine such as N-acetyl galactosamine (NAG), sugars such as glucose, mannose, or the like, carbohydrate, or the like, but is not limited thereto.

According to still another aspect of the present invention, there is provided a method of preparing double-stranded oligo RNA molecules containing the ZBTB7A, YAP1 or CHD1L specific siRNA.

The method of preparing double-stranded oligo RNA molecules containing the ZBTB7A, YAP1 or CHD1L specific siRNA according to the present invention, for example, may include:

(1) binding a hydrophilic compound based on a solid support (the solid support used in the present invention is controlled pore glass (CPG);

(2) synthesizing a RNA single strand based on the solid support (CPG) to which the hydrophilic compound is bound;

(3) covalently binding a hydrophobic compound to a 5′-end of the RNA single strand;

(4) synthesizing a RNA single strand comprising a sequence complementary to that of the RNA single strand;

(5) separating RNA-polymer molecules and the RNA single strand from the solid support (CPG) after synthesizing is completed and then purifying the separated RNA-polymer molecules and RNA single strand; and

(6) preparing double-stranded oligo RNA molecules from the prepared RNA-polymer molecules and the RNA single strand comprising the complementary sequence through annealing.

When the preparation is completed after step (5), whether or not the desired RNA-polymer molecules and the RNA single strand are prepared may be confirmed by measuring molecular weights of the purified RNA-polymer molecules and the RNA single strand using a MALDI-TOF mass spectrometer. In the method, the synthesizing (step (4)) of the RNA single strand comprising the sequence complementary to that of the RNA single strand prepared in step (2) may be performed before step (1) or in any one step of step (1) to step (5).

In addition, the RNA single strand comprising the sequence complementary to that of the RNA single strand synthesized in step (2) may be used in a form in which a phosphate group is bound to the 5′-end.

Meanwhile, there is provided a method of preparing ligand bound-double stranded oligo RNA molecules in which a ligand is additionally bound to the double stranded oligo RNA molecules containing ZBTB7A, YAP1 or CHD1L specific siRNA according to the present invention.

The method of preparing the ligand bound-double-stranded oligo RNA molecules containing the ZBTB7A, YAP1 or CHD1L specific siRNA, for example, may include:

(1) binding a hydrophilic compound to a solid support (CPG) to which a functional group is bound;

(2) synthesizing a RNA single strand onto the solid support (CPG) to which the functional group-hydrophilic compound is bound;

(3) covalently binding a hydrophobic compound to a 5′-end of the RNA single strand;

(4) synthesizing a RNA single strand comprising a sequence complementary to that of the RNA single strand;

(5) separating functional group-RNA-polymer molecules and the RNA single strand comprising complementary sequence from the solid support (CPG) after synthesizing is completed;

(6) binding a ligand to an end of the hydrophilic compound using the functional group to prepare a ligand-RNA-polymer molecule single strand; and

(7) preparing ligand-double-stranded RNA-polymer molecules from the prepared ligand-RNA-polymer molecules and the RNA single strand comprising the complementary sequence through annealing.

When the preparation is completed after step (6), the ligand-RNA-polymer molecules and the RNA single strand comprising the complementary sequence are separated and purified. Then, whether or not the desired ligand-RNA-polymer molecules and the complementary RNA are prepared may be confirmed by measuring molecular weights of the purified RNA-polymer molecules and the RNA single strand using the MALDI-TOF mass spectrometer. The ligand-double-stranded oligo RNA-polymer molecules may be prepared from the prepared ligand-RNA-polymer molecules and the RNA single strand comprising the complementary sequence through annealing. In the method, the synthesizing (step (4)) of the RNA single strand comprising the sequence complementary to that of the RNA single strand prepared in step (3) may be performed as a independent synthetic process before step (1) or in any one step of step (1) to step (6).

According to still another aspect of the present invention, there is provided nanoparticles containing double-stranded oligo RNA molecules comprising ZBTB7A, YAP1 and/or CHD1L specific siRNA.

As described above, the double-stranded oligo RNA molecules comprising ZBTB7A, YAP1 and/or CHD1L specific siRNA are amphiphilic molecules containing both of the hydrophobic and hydrophilic compounds. A hydrophilic part may have affinity for water molecules existing in the body due to interaction such as a hydrogen bond with the water molecule, and the like, to thereby direct toward the outside, and the hydrophobic compounds may direct toward the inside due to the hydrophobic interaction therebetween, thereby forming thermally stable nanoparticles. That is, nanoparticles comprising a form in which the hydrophobic compound is positioned at the center of the nanoparticles and the hydrophilic compound is positioned in a direction toward the outside of the ZBTB7A, YAP1 and/or CHD1L specific siRNA to protect the ZBTB7A, YAP1 and/or CHD1L specific siRNA may be formed. The nanoparticles formed as described above may improve intracellular delivery efficiency of the ZBTB7A, YAP1 and/or CHD1L specific siRNA and effects of the siRNA.

The nanoparticles according to the present invention are characterized in that the nanoparticles are made of the double-stranded oligo RNA molecules comprising siRNAs comprising different sequences. Here, the siRNAs comprising different sequences may be different target genes, for example, ZBTB7A, YAP1 or CHD1L specific siRNA, or be siRNAs comprising different sequences while comprising specificity to the same target gene as each other.

In addition, double-stranded oligo RNA molecules containing another cancer-specific target specific siRNA except for the ZBTB7A, YAP1 or CHD1L specific siRNA may be contained in the nanoparticles according to the present invention.

According to still another aspect of the present invention, there is provided a composition for preventing or treating cancer containing: ZBTB7A, YAP1 or CHD1L specific siRNA; double-stranded oligo RNA molecules containing the same; and/or nanoparticles made of the double-stranded oligo RNA molecules.

The composition containing the ZBTB7A, YAP1 or CHD1L specific siRNA according to the present invention; the double-stranded oligo RNA molecules containing the same; and/or the nanoparticles made of the double-stranded oligo RNA molecules as active ingredients may induce proliferation and apoptosis of cancer cells to thereby exhibit effects of preventing or treating cancer. Therefore, the ZBTB7A, YAP1 or CHD1L specific siRNA according to the present invention and the composition containing the same may be effective in preventing or treating various cancers such as gastric cancer, lung cancer, pancreatic cancer, colon cancer, breast cancer, prostate cancer, ovarian cancer, and kidney cancer as well as liver cancer in which overexpression of the corresponding genes was reported.

Particularly, in the composition for preventing or treating cancer containing double-stranded oligo RNA molecules according to the present invention,

double-stranded oligo RNA molecules containing ZBTB7A specific siRNA composed of a sense strand comprising any one sequence selected from SEQ ID NOs. 1 to 100, preferably, any one sequence selected from the SEQ ID NOs. 1, 2, 3, 4, 5, 6, 7 and 8, more preferably, a sequence of the SEQ ID NOs. 1, 2 or 4, and most preferably, a sequence of the SEQ ID NO. 4 and an antisense strand comprising a sequence complementary to the sense strand, or

double-stranded oligo RNA molecules containing YAP1 specific siRNA composed of a sense strand comprising any one sequence selected from SEQ ID NOs. 101 to 200, preferably, any one sequence selected from the SEQ ID NOs. 103, 107, 108, 112, 116, 117, 118, 121 and 122, more preferably, a sequence of SEQ ID NOs. 116, 118 or 121, and most preferably, a sequence of the SEQ ID NO. 118 and an antisense strand comprising a sequence complementary to the sense strand

double-stranded oligo RNA molecules containing CHD1L specific siRNA composed of a sense strand comprising any one sequence selected from SEQ ID NOs. 201 to 300, preferably, any one sequence selected from the SEQ ID NOs. 201, 202, 203 and 204, more preferably, a sequence of SEQ ID NOs. 202, 203 or 204, and most preferably, a sequence of the SEQ ID NO. 204 and an antisense strand comprising a sequence complementary to the sense strand may be contained.

Alternatively, the double-stranded oligo RNA molecules containing ZBTB7A specific siRNA, the double-stranded oligo RNA molecules containing YAP1 specific siRNA, and the double-stranded oligo RNA molecules containing CHD1L specific siRNA may be included in a mixed form.

In addition, siRNA-specific to another cancer-specific target gene except for the ZBTB7A, YAP1 or CHD1L may be additionally contained in the composition of the present invention.

As described above, in the case of using the composition for preventing or treating cancer containing the double-stranded oligo RNA molecules containing ZBTB7A, YAP1 and CHD1L specific siRNA, or containing the double-stranded oligo RNA molecules containing ZBTB7A, YAP1 and CHD1L specific siRNA and another cancer-specific target specific siRNA, a synergic effect may be obtained like a combination therapy commonly used to treat cancer.

The composition according to the present invention may prevent or treat, for example, liver cancer, gastric cancer, colon cancer, pancreatic cancer, prostate cancer, breast cancer, ovarian cancer, kidney cancer, lung cancer, and the like, but is not limited thereto.

In addition, the nanoparticles contained in the composition for preventing or treating cancer containing nanoparticles made of the double-stranded oligo RNA molecules according to the present invention may be purely composed of any one molecule selected from the double-stranded oligo RNA molecules containing the ZBTB7A, YAP1 and CHD1L specific siRNAs, or comprises double-stranded oligo RNA molecules containing the ZBTB7A, YAP1 and/or CHD1L specific siRNAs in a mixed form.

The composition according to the present invention may be prepared to further contain at least one kind of pharmaceutically acceptable carriers in addition to the active ingredients as describe above. The pharmaceutically acceptable carrier may be compatible with the active ingredients of the present invention, and any one of normal saline, sterile water, Ringer's solution, buffered saline, a dextrose solution, a maltodextrin solution, glycerol, and ethanol or a mixture of at least two thereof may be used. As needed, another general additive such as an antioxidant, a buffer solution, a bacteriostatic agent, or the like, may be added. In addition, the composition may be formulated into a formulation for injection such as an aqueous solution, a suspension, an emulsion, or the like, by additionally adding a diluent, a dispersant, a surfactant, a binder, and a lubricant.

Particularly, the composition may be preferably formulated into a lyophilized formulation.

A method generally known in the art to which the present invention pertains may be used in order to prepare the lyophilized formulation, and a stabilizer for lyophilization may be added. Further, the composition may be preferably formulated using an appropriate method known in the art or a method disclosed in Remington's pharmaceutical Science (Mack Publishing Company, Easton Pa.) according to the disease or the ingredient.

A content and an administration method of the active ingredient contained in the composition according to the present invention may be determined by a person comprising ordinary skill in the art based on patient's symptoms and severity of the disease. In addition, the composition may be formulated into various formulations such as powders, tablets, capsules, liquids, injections, ointments, syrups, and the like, and may be provided in a unit-dose container or multi-dose container, for example, a sealed ampoule, bottle, and the like.

The composition according to the present invention may be orally or parenterally administered. An administration route of the composition according to the present invention is not particularly limited, but oral, intravenous, intramuscular, intraarterial, intramedullary, intradural, intracardiac, transdermal, subcutaneous, abdominal, enteral, sublingual, or local administration may be performed. The dose of the composition according to the present invention may be various according to the weight, the age, the gender, the health status, and the diet of the patient, the administration time, the administration method, the excretion rate, the severity of the disease, or the like, and be easily determined by a person comprising ordinary skill in the art. In addition, the composition may be formulated into an appropriate formulation for clinical administration using a method known in the art.

According to another aspect of the present invention, there is provided a use of ZBTB7A, YAP1 or CHD1L specific siRNA, double-stranded oligo RNA molecules containing the same, and/or nanoparticles made of the double-stranded oligo RNA molecules in the manufacture of a medicament for preventing or treating cancer.

According to still another aspect of the present invention, there is provided a method for preventing or treating cancer including administering the double-stranded oligo RNA molecules according to the present invention, nanoparticles including the double-stranded oligo RNA molecules, and the double-stranded oligo RNA molecules or the nanoparticles to a patient requiring treatment.

### Advantageous Effects

As set forth above, a composition for treating cancer containing ZBTB7A, YAP1 and/or CHD1L specific siRNA according to the present invention or double-stranded oligo RNA molecules containing the same may highly efficiently suppress expression of the ZBTB7A, YAP1 and/or CHD1L gene to effectively treat cancer, particularly, liver cancer without adverse effects, such that the composition may be significantly useful to treat the cancer in which there is no appropriate therapeutic agent.

### BRIEF DESCRIPTION OF DRAWINGS

FIG. 1 is a schematic diagram of a nanoparticle made of a double-stranded oligo RNA molecule according to the present invention;

FIG. 2 is a graph of target gene expression inhibition levels confirmed after transfection of human liver cell line (Huh-7) with siRNAs (0.2, 1, 5 nM) comprising a sequence of SEQ ID NOs. 1 to 8 and SEQ ID No. 310 (ZBTB7A_Ref) according to the present invention as a sense strand;

FIG. 3 is a graph of target gene expression inhibition levels confirmed after transfection of human liver cell line (Huh-7) with the siRNAs comprising the sequences of the SEQ ID NOs. 103, 107, 108, 112, 116 to 118, 121, 122, 301 and SEQ ID No. 311 (YAP_Ref) according to the present invention as a sense strand;
A: target gene expression inhibition levels transfected with the siRNA comprising the sequences of the SEQ ID NOs. 103, 107, 108, 112, 116 to 118, 121, and 301 (0.2, 1, 5 nM)
B: target gene expression inhibition levels transfected with the siRNA comprising the sequences of the SEQ ID NOs. 116 to 118, 121, 122, 301 and 311 (5, 20 nM)

FIG. 4 is a graph of target gene expression inhibition levels confirmed after transfection of human liver cell line (Huh-7) with the siRNAs (0.2, 1, 5 nM) comprising the sequences of the SEQ ID NOs. 201 to 204, 301 and SEQ ID NOs. 312 (CHD1L_Ref) according to the present invention as a sense strand;

FIG. 5 is a graph obtained by confirming inhibition concentrations 50% (IC50s) of ZBTB7A specific siRNA comprising sequences of the SEQ ID NOs. 4 according to the present invention as sense strand
A: IC50 in Hep3B cell line
B: IC50 in Huh-7 cell line

FIG. 6 is a graph obtained by confirming inhibition concentrations 50% (IC50s) of YAP1 specific siRNA comprising sequences of the SEQ ID NOs. 118 according to the present invention as sense strand
A: IC50 in Hep3B cell line
B: IC50 in Huh-7 cell line

FIG. 7 is a graph obtained by confirming inhibition concentrations 50% (IC50s) of CHD1L specific siRNA comprising sequences of the SEQ ID NOs. 204 according to the present invention as sense strand
A: IC50 in Hep3B cell line
B: IC50 in Huh-7 cell line

FIG. 8 is graph showing inhibition effect of siRNAs of the present invention on cell proliferation (Human liver cancer cell line (Huh-7) was treated with siRNAs and control (siCONT))
A: result of siRNA comprising sequences of the SEQ ID NO. 4 according to the present invention as sense strand (5, 20, 100 nM)
B: result of siRNA comprising sequences of the SEQ ID NO. 118 according to the present invention as sense strand (5, 20 nM)
C: result of siRNA comprising sequences of the SEQ ID NO. 204 according to the present invention as sense strand (5, 20, 100 nM)

FIG. 9 is photographs showing colony formation inhibition by corresponding siRNAs through colony forming assay (CFA) after cancer cells are transfected with siRNAs of SEQ ID NOs. 4, 118, and 301 according to the present invention as a sense strand
A: CFA using siRNA of SEQ ID NO. 4
B: CFA using the siRNA of SEQ ID NO. 118

### DETAILED DESCRIPTION OF THE EMBODIMENTS

Hereinafter, the present invention will be described in detail through Examples. However, these Examples are only to illustrate the present invention, and those skilled in the art will appreciate that these Examples are not to be construed as limiting a scope of the present invention.

### Example 1

### Design of Target Sequences of ZBTB7A, YAP1 and CHD1L Gene, and Preparation of siRNA

100 kinds of target sequences (sense strand) capable of binding to an mRNA sequence (NM_015898) of ZBTB7A gene, an mRNA sequence (NM_006106) of YAP1 gene, or an mRNA sequence (NM_004284) of CHD1L gene were designed per each gene, and antisense siRNA strands comprising a sequence complementary to the desired base sequence were prepared. First, the desired base sequence to which the siRNA may bind was designed from the mRNA sequences of the corresponding genes using a Turbo si-Designer developed by Bioneer. siRNA for liver cancer related genes of the present invention has a double-stranded structure composed of a sense strand comprising 19 nucleotides and an antisense strand complementary thereto. In addition, siCONT (SEQ ID NO. 301), which is a siRNA comprising a sequence that does not inhibit expression of genes, was prepared. The siRNA is prepared by connecting a phosphodiester bond configuring a RNA backbone structure using β-cyanoethyl phosphoramidite (Nucleic Acids Research, 12: 4539-4557, 1984). More specifically, a reactant containing RNA comprising a desired length was obtained by repeating a series of processes consisting of deblocking, coupling, oxidation, and capping on a solid support on which nucleotides were adhered using an RNA synthesizer (384 Synthesizer, Bioneer, Korea). The RNA was separated from the reactant and purified using a HPLC (LC918, Japan Analytical Industry, Japan) equipped with a Daisogel C18 (Daiso, Japan) column. Then, whether or not the purified RNA coincides with the desired base sequence was confirmed using a MALDI-TOF mass spectrometer (Shimadzu, Japan). Next, the desired double-stranded siRNAs comprising sense strand of SEQ ID NOs. 1 to 301, 310 to 312 were prepared by binding the sense and antisense RNA stands to each other (See Table 1).

TABLE 1
siRNA sense strand sequence of the present
invention

| SEQ ID No. | Target Gene | Sequence |
|---|---|---|
| 1 | ZBTB7A | CAGACAAGACCUUAAAUGA |
| 2 | ZBTB7A | GUCCGAUGAUGACCUGGAU |
| 3 | ZBTB7A | GACAAGCUGAAGGUGCACA |
| 4 | ZBTB7A | CUCUGAGCGGACGUUAAAA |
| 5 | ZBTB7A | GCAGCUGGACCUUGUAGAU |
| 6 | ZBTB7A | GCUGGACCUUGUAGAUCAA |
| 7 | ZBTB7A | CACAUCUUCUCGUCUCUUU |
| 8 | ZBTB7A | CACUGAGACUUCUUGUCAA |
| 9 | ZBTB7A | CCUCGCAAUAAAACCAACU |
| 10 | ZBTB7A | UGUAACGGAACGGGUACUA |
| 11 | ZBTB7A | CAAAUUCCAAUGUCACAAA |
| 12 | ZBTB7A | CCUUUGCCCACAACUACGA |
| 13 | ZBTB7A | CGGACUCGCCUAAAAACCA |
| 14 | ZBTB7A | GAAUCUAGGGUAGCGCUUU |
| 15 | ZBTB7A | GACAUCCUGAGUGGGCUGA |
| 16 | ZBTB7A | CUGCACUGAGACUUCUUGU |
| 17 | ZBTB7A | ACAAGCUGAAGGUGCACAU |
| 18 | ZBTB7A | AGGCACUGACUGUAAUCCA |
| 19 | ZBTB7A | GACUACUACCUGAAGUACU |
| 20 | ZBTB7A | GAAAACAGAAACCCGAGAA |
| 21 | ZBTB7A | CACUGAGACACAAACCUAU |
| 22 | ZBTB7A | CACAAAUUCCAAUGUCACA |
| 23 | ZBTB7A | CGGAACGGGUACUACACUU |
| 24 | ZBTB7A | GCAUAUGCAAUGCUAGCAU |
| 25 | ZBTB7A | CGCAAUAAAACCAACUCUA |
| 26 | ZBTB7A | GGUUCUGACGUGAAGAGGU |
| 27 | ZBTB7A | GUUACAACCAACUUCUAUG |
| 28 | ZBTB7A | AUAUAUAUGACGCGUCACA |
| 29 | ZBTB7A | GCCCACAACUACGACCUGA |
| 30 | ZBTB7A | CUGCACAGACACCUCAAGA |
| 31 | ZBTB7A | ACUACUACCUGAAGUACUU |
| 32 | ZBTB7A | CUCUGUGACACACACAUCU |
| 33 | ZBTB7A | GCACUGACUGUAAUCCAGG |
| 34 | ZBTB7A | GCUCUGAGCGGACGUUAAA |
| 35 | ZBTB7A | CGGUAACUUCACAGCCGGA |
| 36 | ZBTB7A | CGCCUAAAAACCAAAAAGA |
| 37 | ZBTB7A | CGUUUGGAGAUUCAAAACU |
| 38 | ZBTB7A | GGCAACCAACCACAUUAGA |
| 39 | ZBTB7A | GAACGGGUACUACACUUUA |
| 40 | ZBTB7A | CGGGUACUACACUUUAUCU |
| 41 | ZBTB7A | CAAAGGCACUGACUGUAAU |
| 42 | ZBTB7A | GUGUACGAGAUCGACUUC |
| 43 | ZBTB7A | GUAGAAUCCACUUCUGUUC |
| 44 | ZBTB7A | GCUGCACUGAGACUUCUUG |
| 45 | ZBTB7A | UCAAGAAAGACGGCUGCAA |
| 46 | ZBTB7A | UGCAUAUGCAAUGCUAGCA |
| 47 | ZBTB7A | UGAGCCCUCUUUCCACCUU |
| 48 | ZBTB7A | ACGAGAUCGACUUCGUCAG |
| 49 | ZBTB7A | CCCACAACUACGACCUGAA |
| 50 | ZBTB7A | GGACUCGCCUAAAAACCAA |
| 51 | ZBTB7A | GGGUAGCGCUUUCUCAGAU |
| 52 | ZBTB7A | CUAUGAGUCUUUCAGACAA |
| 53 | ZBTB7A | GGAGAUUCAAAACUUCUGU |
| 54 | ZBTB7A | CCAAUGUCACAAAAGCAAU |
| 55 | ZBTB7A | CGUAGAAUCCACUUCUGUU |
| 56 | ZBTB7A | CUUUAAGGACGAGGACGAG |
| 57 | ZBTB7A | AGAAGAAGAUCCGAGCCAA |
| 58 | ZBTB7A | AGAAUCUAGGGUAGCGCUU |
| 59 | ZBTB7A | GGUACUACACUUUAUCUUA |
| 60 | ZBTB7A | CAGGGCACUUACUAAGGGA |
| 61 | ZBTB7A | GGAGAAAGGAGAUCGGACU |
| 62 | ZBTB7A | CACAUUAGAAGUCUUGGCA |
| 63 | ZBTB7A | CUUGGCACUUUGUAACGGA |
| 64 | ZBTB7A | CUUUGUAACGGAACGGGUA |
| 65 | ZBTB7A | CUCUUUUUACUCAAAGGCA |
| 66 | ZBTB7A | GGUUUUGGUUCCCUUCCCU |
| 67 | ZBTB7A | CUUUCAGACAAGACCUUAA |
| 68 | ZBTB7A | UCUGUGACACACACAUCUU |
| 69 | ZBTB7A | CUGUGACACACACAUCUUC |
| 70 | ZBTB7A | GAGCAGAAGAAAUUCCUAA |
| 71 | ZBTB7A | GUUUGCAUAUGCAAUGCUA |
| 72 | ZBTB7A | ACGUGUACGAGAUCGACUU |
| 73 | ZBTB7A | AGCUGGACCUUGUAGAUCA |
| 74 | ZBTB7A | UCCCUCCUUUCUGACGUUU |
| 75 | ZBTB7A | AGAGUCACGAUCAGAGGAA |
| 76 | ZBTB7A | CCAACGUGGGUGACAUCCU |
| 77 | ZBTB7A | AGGGCACUUACUAAGGGAG |
| 78 | ZBTB7A | AGCCGGACUCGCCUAAAAA |
| 79 | ZBTB7A | AACGGGUACUACACUUUAU |
| 80 | ZBTB7A | CAAGACCUUAAAUGAUUUC |
| 81 | ZBTB7A | UGCAAGACCUUCGUCCGCU |
| 82 | ZBTB7A | GCACUUUAAGGACGAGGAC |
| 83 | ZBTB7A | GAGAAAGGAGAUCGGACUG |
| 84 | ZBTB7A | GCACUUUGUAACGGAACGG |
| 85 | ZBTB7A | ACGGAACGGGUACUACACU |
| 86 | ZBTB7A | ACGUAGAAUCCACUUCUGU |
| 87 | ZBTB7A | CAUUGUUAAAGGGAAGCUU |
| 88 | ZBTB7A | ACAUCUGCAAGGUCCGCUU |
| 89 | ZBTB7A | UGAAGGUGCACAUGCGGAA |
| 90 | ZBTB7A | ACUUCUGUCUUCGUCCUCU |
| 91 | ZBTB7A | AGGCAACAGUGUGGGAUAA |
| 92 | ZBTB7A | UUAGAAGUCUUGGCACUUU |
| 93 | ZBTB7A | ACUGAGACUUCUUGUCAAU |
| 94 | ZBTB7A | ACAUCUUCUCGUCUCUUUU |
| 95 | ZBTB7A | CUCAAGAAAGACGGCUGCA |
| 96 | ZBTB7A | GGCAACAGUGUGGGAUAAA |
| 97 | ZBTB7A | AAAAGGCAACCAACCACAU |
| 98 | ZBTB7A | GCUUGGGCCGGUUGAAUGU |
| 99 | ZBTB7A | UCCUCCCUAGCUCAGGGAU |
| 100 | ZBTB7A | GCACAGACACCUCAAGAAA |
| 101 | YAP1 | CAGAAGAUCAAAGCUACUU |
| 102 | YAP1 | GUGCUAUCAUUAGUCACAU |
| 103 | YAP1 | CAGGAAUUGAGAACAAUGA |
| 104 | YAP1 | GUGAGUAGGUUCAUAAUGU |
| 105 | YAP1 | GAACAAAACGAGCAUGAAU |
| 106 | YAP1 | CUCAGACUUAGAAGUCAGA |
| 107 | YAP1 | CUCUUCAACGCCGUCAUGA |
| 108 | YAP1 | GAGUACAGACAGUGGACUA |
| 109 | YAP1 | GAAUUGUGGGUGUGCCUAU |
| 110 | YAP1 | CUUGGAAGGAGUGCCUAUA |
| 111 | YAP1 | GUAGCCACAGAUUAAGAUU |
| 112 | YAP1 | CGAGAUGAGAGUACAGACA |
| 113 | YAP1 | GGUUUACCUUCAUUUAGCU |
| 114 | YAP1 | CAGAUGGAGUUUUAGAGUA |
| 115 | YAP1 | GAGAUGAGAGUACAGACAG |
| 116 | YAP1 | GAGUUCUGACAUCCUUAAU |
| 117 | YAP1 | GAGAUGGAUACAGGUGAUA |
| 118 | YAP1 | GCUGCCACCAAGCUAGAUA |
| 119 | YAP1 | GUACUUUCAGUGCUCAAAA |
| 120 | YAP1 | CCUCGCAAGCAUGUUGUUA |
| 121 | YAP1 | AGAUGAGAGUACAGACAGU |
| 122 | YAP1 | GAGAUGGAAUGAACAUAGA |
| 123 | YAP1 | GACAGUCUUCUUUUGAGAU |
| 124 | YAP1 | GACAGUGGACUAAGCAUGA |
| 125 | YAP1 | GUUGUUUCUUCAGCUUCCU |
| 126 | YAP1 | CGAGCAUGAAUUAACUCUU |
| 127 | YAP1 | CUGUGAUACCUGGCACAGU |
| 128 | YAP1 | GGAGACCUAAGAGUCCUUU |
| 129 | YAP1 | GUUUGAAUCAUAGCCUUGA |
| 130 | YAP1 | CAAAAGUGGGUGGCAAUAU |
| 131 | YAP1 | GAUGAAUUGGAAAGGAGCA |
| 132 | YAP1 | GCCUUAGUUUUGGAAGUAA |
| 133 | YAP1 | GGAAGUGACUUUGCUACAA |
| 134 | YAP1 | GCUCAUAUGUUAGGUACUU |
| 135 | YAP1 | CUAGUUUGUAGUUCUCAUU |
| 136 | YAP1 | GCUGCCAUUAAAGGCAGCU |
| 137 | YAP1 | GGCAUGAGACAAUUUCCAU |
| 138 | YAP1 | CCUUGAUGUGGUCUCUUGU |
| 139 | YAP1 | CCUGCGUAGCCAGUUACCA |
| 140 | YAP1 | GACUCAAAAUCCAGUGUCU |
| 141 | YAP1 | CAAGUCUGCAGGAAGCUUU |
| 142 | YAP1 | GGAAGUGAGCCUGUUUGGA |
| 143 | YAP1 | GCUUUAUAGUGGUUUACCU |
| 144 | YAP1 | GCAUGCUCAUAUGUUAGGU |
| 145 | YAP1 | GCACCUAUCACUCUCGAGA |
| 146 | YAP1 | GGACUAAGCAUGAGCAGCU |
| 147 | YAP1 | GAGUUUGAAUCAUAGCCUU |
| 148 | YAP1 | GGUGGAUUUUAUCCUCGCA |
| 149 | YAP1 | CAUAAGCCAGUUGCAGUUU |
| 150 | YAP1 | GUGUCUACAGGAGUAAUAA |
| 151 | YAP1 | UCAUGUCACAGCAUUUAGU |
| 152 | YAP1 | UGUCCUUGUUCCUAAUGUA |
| 153 | YAP1 | UCAGUCAGGGCUUCUUAGA |
| 154 | YAP1 | UCACUCUCGAGAUGAGAGU |
| 155 | YAP1 | UGAAGGAUCUAAGGAGACA |
| 156 | YAP1 | GAGUAAUAAUGGUUUCCAA |
| 157 | YAP1 | UAUUUUGGCCCUUCAAUUU |
| 158 | YAP1 | GAGAUGGCAAAGACAUCUU |
| 159 | YAP1 | CAGCAGAAUAUGAUGAACU |
| 160 | YAP1 | CACCAAGCUAGAUAAAGAA |
| 161 | YAP1 | GCACCGGAAAUUUCCAUAA |
| 162 | YAP1 | CCAGUGGAAAAACAUGAUU |
| 163 | YAP1 | GAUUAUCUGCUCUCUCUUU |
| 164 | YAP1 | GUCCUUGUUCCUAAUGUAA |
| 165 | YAP1 | ACAGCAUGUUCGAGCUCAU |
| 166 | YAP1 | CUAGAAUAAGCCCUUAUUU |
| 167 | YAP1 | GUCUCAGGAAUUGAGAACA |
| 168 | YAP1 | CUAAAUCUGUGAAGGAUCU |
| 169 | YAP1 | GAACAAACGUCCAGCAAGA |
| 170 | YAP1 | GUGUUCUAGAAAGAGCUAU |
| 171 | YAP1 | CAUAAUGUGCAUGACAGAA |
| 172 | YAP1 | CACCUAAGUACACCCACAA |
| 173 | YAP1 | GAUGUAAGAGCAUGCUCAU |
| 174 | YAP1 | GGAUGGUGGGACUCAAAAU |
| 175 | YAP1 | GGGCAUACGGUAGAUAUUA |
| 176 | YAP1 | CUAGCACCUCUGUGUUUUA |
| 177 | YAP1 | GGAAGGAGUGCCUAUAAUU |
| 178 | YAP1 | GAAGGAGUGCCUAUAAUUU |
| 179 | YAP1 | UCACCUAAGUACACCCACA |
| 180 | YAP1 | UGAGAUACCUGAUGAUGUA |
| 181 | YAP1 | UCAGGGCUUCUUAGAUCUA |
| 182 | YAP1 | AGAUGGAGUUUUAGAGUAG |
| 183 | YAP1 | CGACAGUCUUCUUUUGAGA |
| 184 | YAP1 | GAAUUGAGAACAAUGACGA |
| 185 | YAP1 | CUCUGUGUUUUAAGGGUCU |
| 186 | YAP1 | CUAGAAUGCAAAAUUGGGU |
| 187 | YAP1 | GUGGAUUUUAUCCUCGCAA |
| 188 | YAP1 | CCUACUUCUAUGCUGAAAA |
| 189 | YAP1 | GGAACAAAACGAGCAUGAA |
| 190 | YAP1 | GCAAUCACUGUGUUGUAUA |
| 191 | YAP1 | CCUAAGUACACCCACAAAA |
| 192 | YAP1 | GGCUUCUUAGAUCUACUUA |
| 193 | YAP1 | ACCGUUUCCCAGACUACCU |
| 194 | YAP1 | UGGAAUGAACAUAGAAGGA |
| 195 | YAP1 | UUGCUCUUCCUUGUCCAUU |
| 196 | YAP1 | UCUUACGAUGCCCUCUGUA |
| 197 | YAP1 | CUAUGAAGUAAUAGUUGGU |
| 198 | YAP1 | CAGUUUUCAGGCUAAUACA |
| 199 | YAP1 | CUCAGCUUGGGAAGAUAGA |
| 200 | YAP1 | CAGUCAGGGCUUCUUAGAU |
| 201 | CHD1L | UUCUUACUGCGGCUUCAUA |
| 202 | CHD1L | CUGGAUAAGCUACUAGCAU |
| 203 | CHD1L | GGAGCCUUUUGAAGUUGGA |
| 204 | CHD1L | CUGCUGCAUAAGACCUUGU |
| 205 | CHD1L | CUGAGUCAGCAAGUGAACU |
| 206 | CHD1L | GAGCUUCCCAAGAAGACAG |
| 207 | CHD1L | CGUUCCAAUGUCCUGUCUG |
| 208 | CHD1L | CCUCCUCAAGACAGCUGGU |
| 209 | CHD1L | GGUGGGAAUCCAACAAUUA |
| 210 | CHD1L | GCAUCCCAACUUACAUAUA |
| 211 | CHD1L | CUCAUCGCAUUGGCCAAAA |
| 212 | CHD1L | GAAGAAAGCAAGUGUUCAU |
| 213 | CHD1L | AGUGUUCAUCUUCCACGUA |
| 214 | CHD1L | GUCACGUUUUCAUGUGCUA |
| 215 | CHD1L | GAGUGUUCUUGUUGUGGAU |
| 216 | CHD1L | GCUCAGCAUCGUGAUCGUU |
| 217 | CHD1L | CUUGGCCAUUAAGAACUUU |
| 218 | CHD1L | GAUGAAGCUCACAGGUUGA |
| 219 | CHD1L | CCUUGUCAGAGUUCUCAGU |
| 220 | CHD1L | CUGAAACAGGAGUCACGUU |
| 221 | CHD1L | CACGUUUUCAUGUGCUACU |
| 222 | CHD1L | CAGCAAGUGAACUGCACAA |
| 223 | CHD1L | GUGAUAUACCAUGGCAUGU |
| 224 | CHD1L | CUUUGGACAGCAGCCCAUU |
| 225 | CHD1L | GAGGUACUGCAAUAGAGUA |
| 226 | CHD1L | CUCAGAAUGACUUGCAAGC |
| 227 | CHD1L | CAAGACCUGAAACAGGAGU |
| 228 | CHD1L | CCUCAAGUACGUUAGUGGU |
| 229 | CHD1L | CUUUGUCCCUUGUCUGUUU |
| 230 | CHD1L | AGAAGGAGGCCAUUUUACU |
| 231 | CHD1L | AGAAAGCAAGUGUUCAUCU |
| 232 | CHD1L | GGGCAAGAUUUGUUGGCCU |
| 233 | CHD1L | GAAAAUGAGACGGCAAAGA |
| 234 | CHD1L | GGAGCACCAUGGAUGAAAU |
| 235 | CHD1L | CCUGGUGGGAAUCCAACAA |
| 236 | CHD1L | UUGAAAAUGAGACGGCAAA |
| 237 | CHD1L | UGAUUGGUCGAGACACUGU |
| 238 | CHD1L | AGUUGAGUGAGAUACUCAA |
| 239 | CHD1L | GUGAACUGCACAAACUCUU |
| 240 | CHD1L | CUCCAAGACUAUAUGGAUU |
| 241 | CHD1L | CACUUGGCCAUUAAGAACU |
| 242 | CHD1L | GUGUUCAUCUUCCACGUAU |
| 243 | CHD1L | UCAGCAAGUGAACUGCACA |
| 244 | CHD1L | GGCCGAUCACUCCGAAAUA |
| 245 | CHD1L | CCGAUCACUCCGAAAUAAA |
| 246 | CHD1L | GGCAGAGGUGGUUUAUUUA |
| 247 | CHD1L | GGGAGGUGUCCUUUUAUUU |
| 248 | CHD1L | GACCUUGUCAGAGUUCUCA |
| 249 | CHD1L | GUGGUUUAUUUACAGCUCU |
| 250 | CHD1L | GCUUGAAAGAUGCAUCAUU |
| 251 | CHD1L | CCCAAAUGACCCAGAUGUU |
| 252 | CHD1L | CAAGACCCAGAUGCUACUU |
| 253 | CHD1L | CAGCUUGCUAGUUGCAUAA |
| 254 | CHD1L | UGAACAACUGGUAAACCUU |
| 255 | CHD1L | UCAUUGUGCACUGCGUAGA |
| 256 | CHD1L | AGGUUUUAACUGGUAUGGU |
| 257 | CHD1L | CUGCAAUAGAGUAUUUCAA |
| 258 | CHD1L | GACUACCUAUGAGAUUUGC |
| 259 | CHD1L | CGAGGAUGCUCUCAUUGUG |
| 260 | CHD1L | GAUUUGUUGGCCUUGAUUG |
| 261 | CHD1L | GCUUCUUACUGCGGCUUCA |
| 262 | CHD1L | GCUGACAGGGAUUCACCUA |
| 263 | CHD1L | CCCUGCUGCAUAAGACCUU |
| 264 | CHD1L | GCUUCGAAAGUGUGUGGAU |
| 265 | CHD1L | GCUGAUUGGUCGAGACACU |
| 266 | CHD1L | GCCAAGAGAAGGAGACUCA |
| 267 | CHD1L | CUGCAUAAGACCUUGUCAG |
| 268 | CHD1L | GCUGGAUAAGCUACUAGCA |
| 269 | CHD1L | CUCCGAAAUAAAGGCAGUG |
| 270 | CHD1L | CCAUUAAGAACUUUGGACA |
| 271 | CHD1L | CAGAAAACCCUUUUGGAGA |
| 272 | CHD1L | GGAGUGUUCUUGUUGUGGA |
| 273 | CHD1L | GGAUGGUUCUGUGAGAGGA |
| 274 | CHD1L | CAGCUGGAGGGAGUAAACU |
| 275 | CHD1L | GUUGAGUGAGAUACUCAAA |
| 276 | CHD1L | GAUUAUUCUAAAGAGCCCA |
| 277 | CHD1L | CAGAGGUGGUUUAUUUACA |
| 278 | CHD1L | GCAUUAAGAUGGCAGCCCU |
| 279 | CHD1L | UCACCAACAUGAUCAUAGA |
| 280 | CHD1L | AGUUGGAGACCACCUGACU |
| 281 | CHD1L | UGGUCGAGACACUGUGGAA |
| 282 | CHD1L | AACAAUUACCAGUCCUUCU |
| 283 | CHD1L | CCUACGCUCUUACCAGCUG |
| 284 | CHD1L | CUGACUACCUAUGAGAUUU |
| 285 | CHD1L | GGUGGGAGAUUUUAUUCAA |
| 286 | CHD1L | GACCUAGAUGCAUUUGAAA |
| 287 | CHD1L | CACCAACAUGAUCAUAGAA |
| 288 | CHD1L | CCUCCAGUUGAGUGAGAUA |
| 289 | CHD1L | CUGGUAAACCUUCAGAAAA |
| 290 | CHD1L | GCUCUGCUGAGCUGGAUUA |
| 291 | CHD1L | CCGAGGAUGCUCUCAUUGU |
| 292 | CHD1L | CAAUGUCCUGUCUGGCAUU |
| 293 | CHD1L | AUAGAAGGAGGCCAUUUUA |
| 294 | CHD1L | ACUGCAAUAGAGUAUUUCA |
| 295 | CHD1L | CAGUUGAGUGAGAUACUCA |
| 296 | CHD1L | CCUUAAGAAUUGGCCCAGC |
| 297 | CHD1L | UCCCUGCUGCAUAAGACCU |
| 298 | CHD1L | GCUUGCUAGUUGCAUAAUA |
| 299 | CHD1L | GCUGGUGCCUUAAGAAUUG |
| 300 | CHD1L | AACUUUGGACAGCAGCCCA |
| 301 | siCONT | CUUACGCUGAGUACUUCGA |
| 310 | ZBTB7A_Ref | GGGCGUCAUGGACUACUAC |
| 311 | YAP1_Ref | GACAUCUUCUGGUCAGAGA |
| 312 | CHD1L_Ref | CGUAUUGGACAUGCCACGAAA |

### Example 2

### Preparation of Double-Stranded Oligo RNA Molecules (SAMiRNA LP)

The double-stranded oligo RNA molecules (SAMiRNA LP) prepared in the present invention had a structure of the following Structural Formula (5).

C₂₄-5′-S-3′-PEG

AS Structural Formula (5)

In Structural Formula (5), S is a sense strand of siRNA; AS is an antisense strand of the siRNA; PEG is a polyethylene glycol as a hydrophilic compound; C₂₄ is tetradocosane including a disulfide bond as a hydrophobic compound; and 5′ and 3′ mean orientations of ends of the double-stranded oligo RNA.

In the case of the sense strand of the siRNA in Structural Formula (5), oligo RNA-hydrophilic compound molecule comprising a sense strand of which polyethylene glycol was bound to a 3′-end region was synthesized by a method of connecting the phosphodiester bond configuring the backbone structure of the RNA using β-cyanoethyl phosphoramidite as described above while using polyethylene glycol (PEG, Mn=2,000)-CPG prepared according to a method in Example 1 disclosed in the existing Patent (KR 2012-0119212A) as the support, and then tetradocosane including the disulfide bond was bound to a 5′-end region, thereby preparing a sense strand of the desired RNA-polymer molecules. In the case of the antisense strand to be annealed with the strand, the antisense strand comprising the sequence complementary to that of the sense strand was prepared by the above-mentioned reaction.

After synthesizing was completed, the synthesized RNA single strand and the RNA polymer molecules were separated from the CPG by treating the reactants with ammonia (28% (v/v)) in a water bath at 60° C., and then a protective residue was removed by a deprotection reaction. The RNA single strand and the RNA polymer molecules from which the protective residue was removed were treated with N-methylpyrrolidone, triethylamine, and triethylaminetrihydrofluoride at a volume ratio of 10:3:4 in an oven at 70° C., thereby removing tert-butyldimethylsilyl (2′TBDMS).

The RNA was separated from the reactant and purified using a HPLC (LC918, Japan Analytical Industry, Japan) equipped with a Daisogel C18 (Daiso, Japan) column. Then, whether or not the purified RNA coincides with the desired base sequence was confirmed using a MALDI-TOF mass spectrometer (Shimadzu, Japan). Thereafter, in order to prepare each of the double-stranded oligo RNA polymer molecules, the same amount of sense and antisense strands were mixed and put into 1× annealing buffer (30 mM HEPES, 100 mM potassium acetate, 2 mM magnesium acetate, pH 7.0-7.5), followed by reacting with each other in a water bath at 90° C. for 3 minutes and reacting with each other again at 37° C., thereby preparing the double-stranded oligo RNA molecules containing siRNAs of the SEQ ID NOs. 1 to 8, 103, 107, 108, 112, 116 to 118, 121, 122, 201 to 204 and 301 as a sense strand, respectively (hereinafter, referred to as SAMiRNALP-ZBTB, SAMiRNALP-YAP, SAMiRNALP-CHD, SAMiRNALP-CONT, respectively). It was confirmed through electrophoresis that the prepared double-stranded oligo RNA molecules were annealed.

### Example 3

### Preparation of Nanoparticles (SAMiRNA) Made of SAMiRNA LP and Measurement of Size

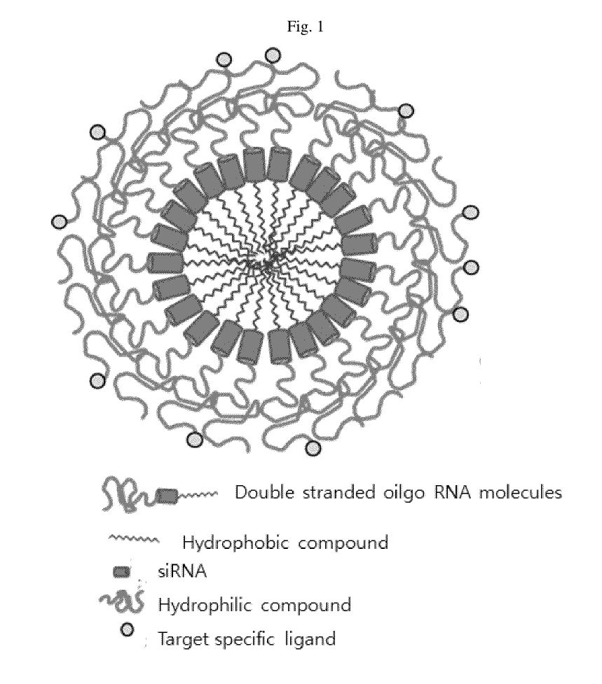

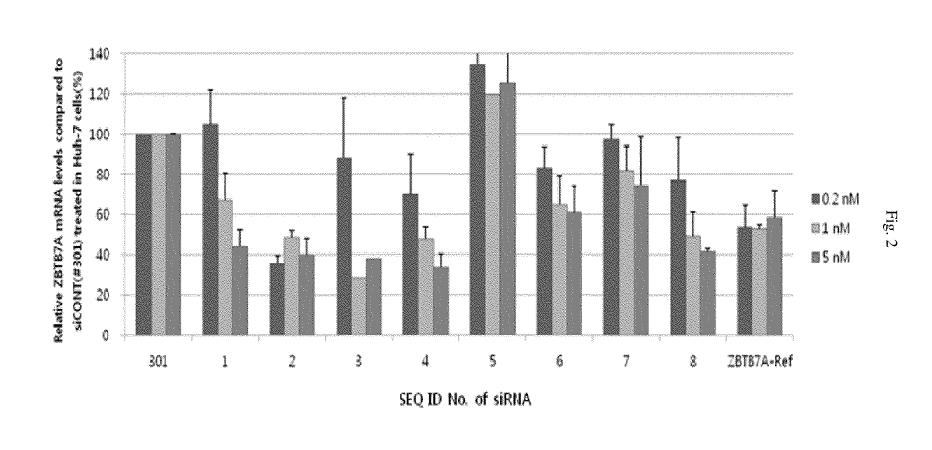

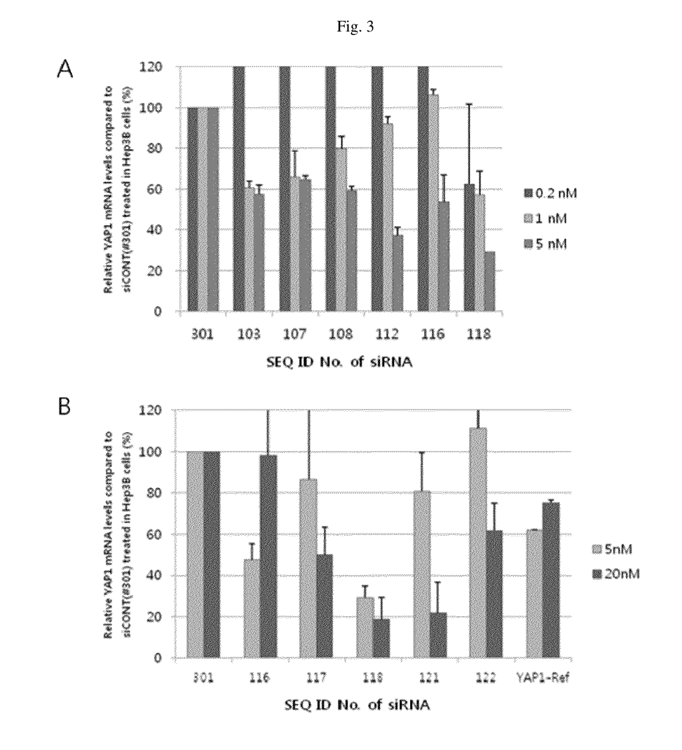

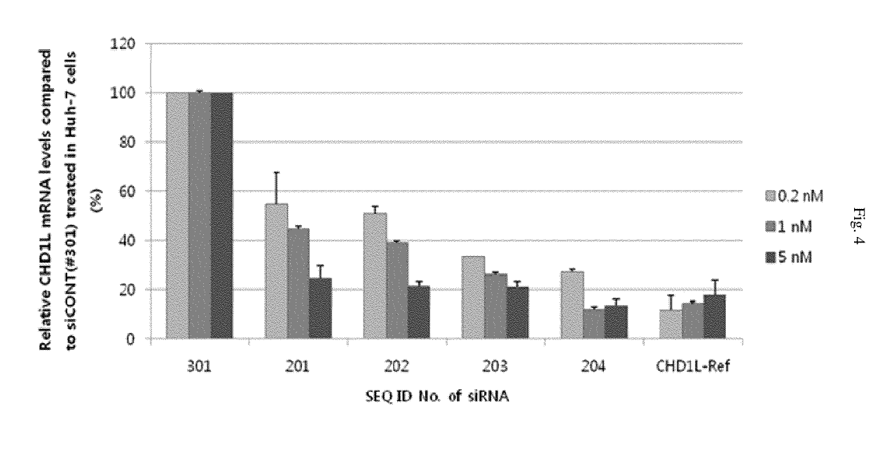

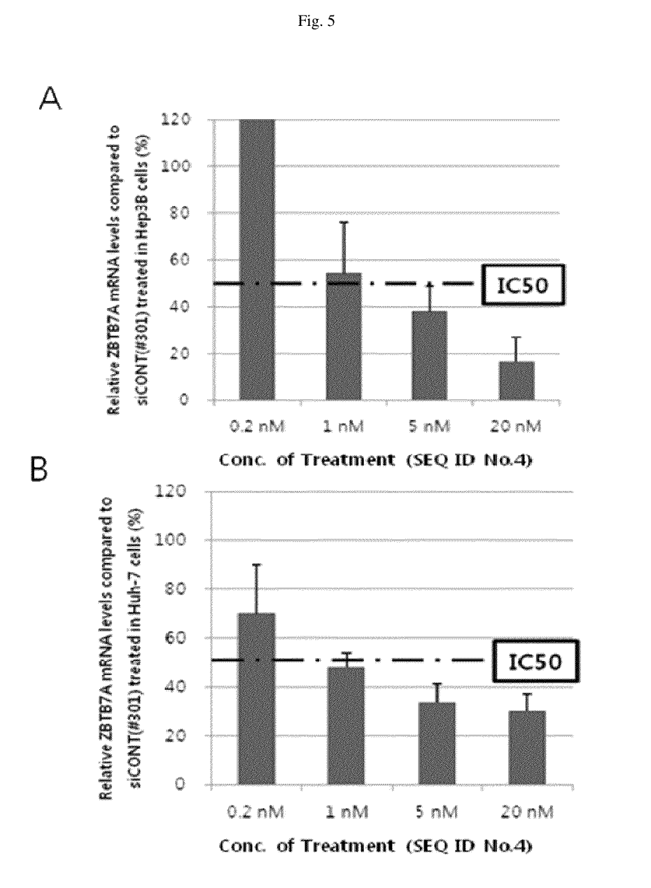

The SAMiRNA LP prepared in Example 2 formed nanoparticles, that is, micelles by hydrophobic interactions between the hydrophobic compounds bound to the ends of the double-stranded oligo RNA (See FIG. 1).

Sizes and polydispersity indexes (PDI) of nanoparticles made of SAMiRNALP-ZBTB, SAMiRNALP-YAP, SAMiRNALP-CHD, and SAMiRNALP-CONT, respectively were analyzed, thereby confirming formation of the nanoparticles (SAMiRNA) made of the corresponding SAMiRNALP.

### Example 3-1

### Preparation of Nanoparticles

After dissolving SAMiRNALP-ZBTB in 1.5 ml, Dulbecco's Phosphate Buffered Saline (DPBS) at a concentration of 50 μg/ml, nanoparticle powder was prepared by lyophilization at −75° C. and 5 mTorr for 48 hours and dissolved in the DPBS as a solvent, thereby preparing homogeneous nanoparticles. SAMiRNALP-YAP, SAMiRNALP-CHD and SAMiRNALP-CONT were prepared by using the same method.

### Example 3-2

### Measurement of Sizes and Polydispersity Indexes (Hereinafter, Referred to as ‘PDI’) of Nanoparticles

The sizes of the nanoparticles were measured using a zeta-potential measurement. The sizes of the homogeneous nanoparticles prepared in Example 3-1 were measured using the zeta-potential measurement (Nano-ZS, MALVERN, UK). Here, a refractive index and absorption index for compounds were set to 1.459 and 0.001, respectively. In addition, a temperature of DPBS as the solvent was input as 25° C., and viscosity and a refractive index thereof were input as 1.0200 and 1.335, respectively. A one-time measurement consists of 15 repetitive size measurements, and this measurement was repeated six times.

### Example 4

### Confirmation of Target Gene Expression Inhibition in Human Liver Cancer Cell Lines (Hep3B and Huh-7 Cell Lines) Using the siRNAs

The human liver cancer cell lines (Huh-7 cell lines) were transfected using the siRNAs comprising sense strand of the SEQ ID NOs. 1 to 8, 103, 107, 108, 201 to 204 and 301 prepared in Example 1, respectively, and expression levels of the target genes in the transfected Huh-7 cell lines were analyzed.

The human liver cancer cell lines (Hep3B cell lines) were transfected using the siRNAs comprising sense strand of the SEQ ID NOs. 112, 116 to 118, 121, 122 and 301 prepared in Example 1, respectively, and expression levels of the target genes in the transfected Hep3B cell lines were analyzed.

### Example 4-1

### Culture of Human Liver Cancer Cell Lines

The human liver cancer cell lines (Hep3B and Huh-7 cell lines) obtained from American Type Culture Collection (ATCC) were cultured in an Eagle's minimum essential medium (EMEM, GIBCO/Invitrogen, USA) supplemented with 10% (v/v) fetal bovine serum, 100 units/ml penicillin, and 100 μg/ml streptomycin at 37° C. under 5% (v/v) CO₂ atmosphere.

### Example 4-2

### Transfection of the Desired siRNA in Human Liver Cancer Cell Lines

After 1×10⁵ Hep3B cell lines and 1×10⁵ Huh-7 cell lines cultured in the Example 4-1 were cultured in a 12-well plate using the EMEM at 37° C. under 5% (v/v) CO₂ atmosphere for 18 hours, the medium was removed, and then 500 μl of Opti-MEM medium (GIBCO, US) was dispensed in each well.

Meanwhile, 1.5 μl of Lipofectamine™ RNAi Max (Invitrogen, US) and 248.5 μl of the Opti-MEM medium were mixed with each other to prepare a mixed solution and then reacted with each other at room temperature for 5 minutes. Then, 0.2, 1 or 5 μl of each of the siRNAs (1 pmole/μl) comprising SEQ ID NOs. 1 to 8 and 310 (ZBTB7A-Ref (J Biomed Biotechnol. 2009; 2009:514287)) for ZBTB7A, SEQ ID Nos. 103, 107, 108, 112, 116 to 118, 121, 122 and 311 (YAP1-Ref (Molecular Cell, Vol. 11, 11-23, January, 2003)) for YAP1, and SEQ ID Nos. 201 to 204 and 312 (CHD1L-Ref (J Clin Invest 2010 Apr. 1; 120(4): 1178-91)) for CHD1L as a sense strand prepared in Example 1 was added to 230 μl of the Opti-MEM medium, thereby preparing a siRNA solution comprising a final concentration of 0.2, 1, 5 or 20 nM. The Lipofectamine™ RNAi Max mixed solution and the siRNA solution were mixed and then reacted with each other at room temperature for 20 minutes, thereby preparing a solution for transfection.

Thereafter, 500 μl of the solution for transfection was dispensed in each well containing tumor cell lines and the dispensed Opti-MEM medium and cultured for 6 hours, followed by removal of the Opti-MEM medium. Here, 1 ml of the EMEM medium was dispensed in each well and cultured at 37° C. under 5% (v/v) CO₂ atmosphere for 24 hours.

### Example 4-3

### Quantitative Analysis of Target Gene mRNA

Total RNA was extracted from the cell lines transfected in the example 4-2 to prepare cDNA, and then a target gene mRNA expression level was relatively quantified using a real-time polymerase chain reaction (PCR).

### Example 4-3-1

### Separation of RNA from Transfected Cells and Preparation of cDNA

Total RNA was extracted from the cell lines transfected in the example 4-2 by using an RNA extraction kit (AccuPrep Cell total RNA extraction kit, Bioneer, Korea), and cDNA was prepared from the extracted RNA using an RNA reverse transcriptase (AccuPower CycleScript RT Premix/dT20, Bioneer, Korea), as follows. More specifically, 1 μg of the extracted RNA was put into each of the 0.25 ml Eppendorf tubes containing AccuPower CycleScript RT Premix/dT20 (Bioneer, Korea), and distilled water treated with diethyl pyrocarbonate (DEPC) was added so as to have a total volume of 20 μl. Two steps of RNA-primer hybridization at 30° C. for 1 minute and preparation of cDNA at 52° C. for 4 minutes were repeated six times using a PCR machine (MyGenie™ 96 Gradient Thermal Block, Bioneer, Korea), and then the amplification reaction was terminated by inactivating enzymes at 95° C. for 5 minutes.

### Example 4-3-2

### Relative Quantitative Analysis of Target Gene mRNA

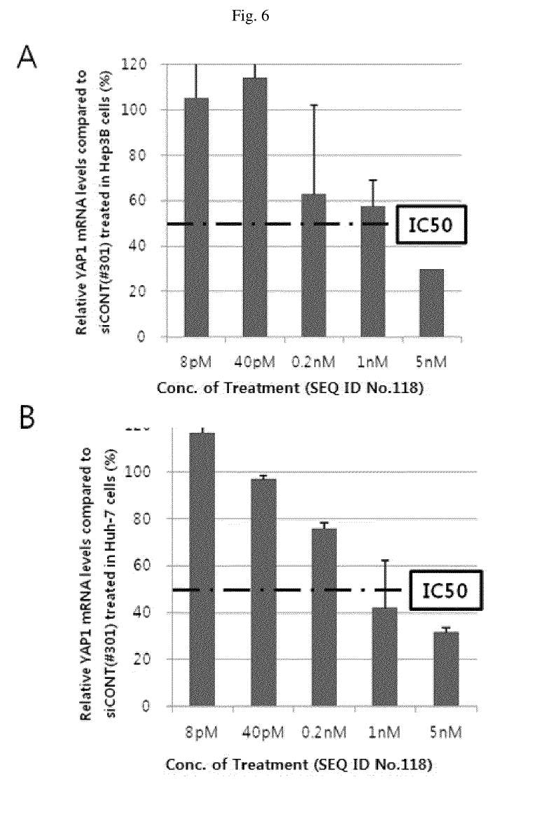

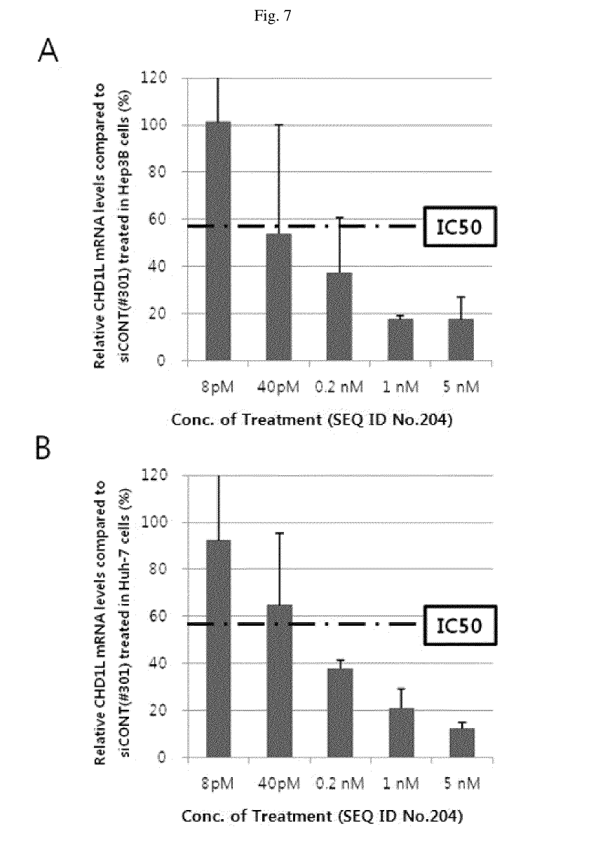

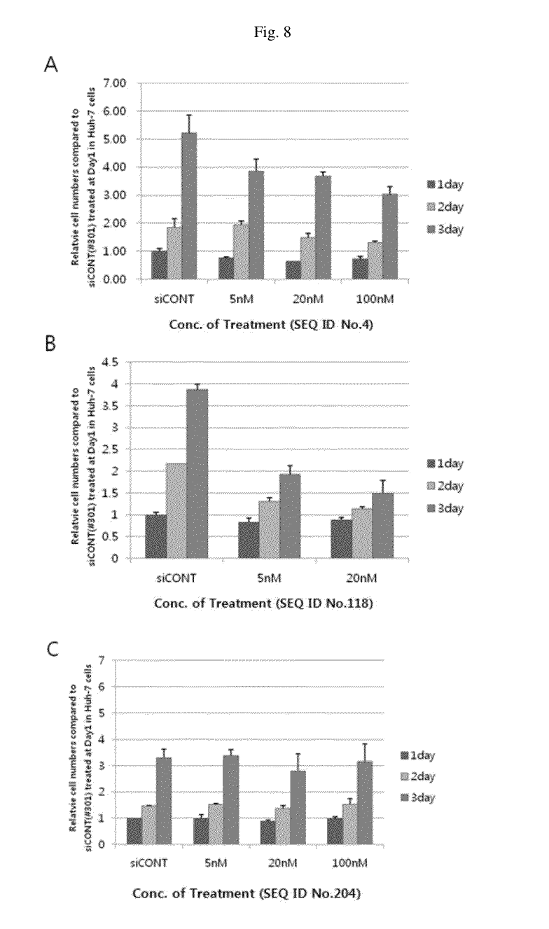

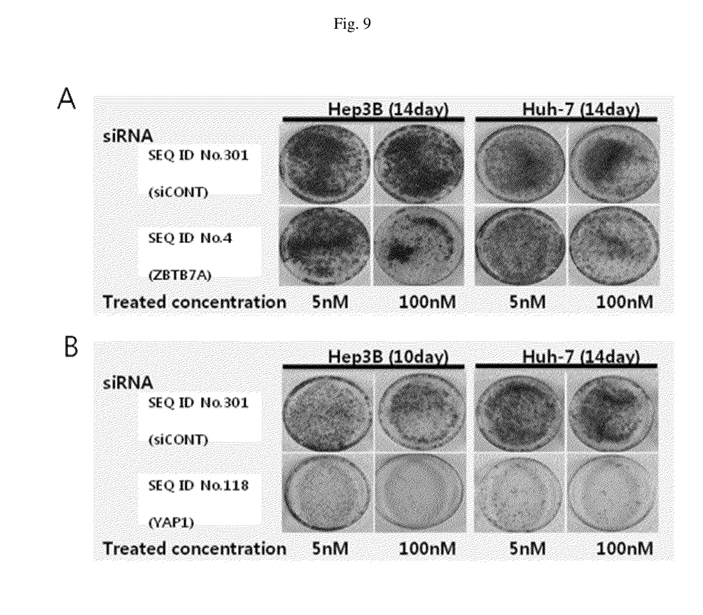

The relative level of liver cancer related gene mRNA was quantified through the real-time PCR using the cDNA prepared in the example 4-3-1 as a template as follows. The cDNA prepared in the example 4-3-1 was diluted 5 times with distilled water in each well of a 96-well plate, and then in order to accurately analyze the target gene mRNA expression level, 3 μl of the diluted cDNA, 10 μl of 2× GreenStar™ PCR master mix (Bioneer, Korea), 6 μl of distilled water, and 1 μl of ZBTB7A qPCR primers (each of F and R: 10 pmole/μl, Bioneer, Korea, See Table 2) were used to prepare a mixed solution. Meanwhile, in order to normalize the target gene mRNA expression level, glyceraldehyde 3-phosphate dehydrogenase (GAPDH), which is a housekeeping gene (hereinafter, referred to as HK gene), was used as a reference gene. The following reaction was performed on the 96-well plate containing the mixed solution using an Exicycler™96 Real-Time Quantitative Thermal Block (Bioneer, Korea). Enzyme activation and a secondary structure of cDNA were removed by performing the reaction at 95° C. for 15 minutes. Then, four steps of denaturing at 94° C. for 30 seconds, annealing at 58° C. for 30 seconds, extension at 72° C. for 30 seconds, and SYBR green scan were repetitively performed 42 times, and then a final extension was performed at 72° C. for 3 minutes. Thereafter, the temperature was maintained at 55° C. for 1 minute, and a melting curve of 55° C.-95° C. was analyzed. After finishing the PCR, each of the obtained threshold cycle (Ct) values of the target genes was corrected using the GAPDH gene, thereby obtaining the corrected Ct value of the target gene. Then, a difference (ΔCt) in Ct value was calculated using an experimental group treated with the siRNA(siCONT) comprising a control sequence that does not inhibit gene expression as a control group. The expression levels of the target genes in the cells treated with ZBTB7A specific siRNAs comprising sense strand of SEQ ID NOs. 1 to 8 and ZBTB7A-Ref were relatively quantified, respectively, using the ΔCt values and the calculation equation of 2(−ΔCt)×100 (See FIG. 2).

In addition, in each of the experimental groups treated with YAP1 or CHD1L specific siRNAs (SEQ ID NOs. 103, 107, 108, 112, 116 to 118, 121, 122, YAP1-Ref, 201 to 204 and CHD1L-Ref), mRNA of the target gene was relatively quantified by the same method using the YAP1 or CHD1L qPCR primer and the GAPDH qPCR primer (FIGS. 3 and 4).

In order to select the siRNA comprising high efficiency, the siRNAs used in the case in which the mRNA expression levels for each gene at the concentrations of 0.2 nM and 1 nM were commonly significantly decreased were selected (SEQ ID NOs. 4, 118, and 204 as a sense strand).

TABLE 2
qPCR primer sequence information
(F: forward primer, R: reverse primer)

| name | Sequence | SEQ ID NO. |
|---|---|---|
| GAPDH-F | GGTGAAGGTCGGAGTCAACG | 302 |
| GAPDH-R | ACCATGTAGTTGAGGTCAATGAAGG | 303 |
| ZBTB7A-F | AGTGCTTCTCCTGGCCGTTG | 304 |
| ZBTB7A-R | CGACCACCTGCACAGACACC | 305 |
| YAP1-F | GAACCGTTTCCCAGACTACC | 306 |
| YAP1-R | GCATCAGCTCCTCTCCTTCT | 307 |
| CHD1L-F | CCGATCACTCCGAAATAAA | 308 |
| CHD1L-R | GCCTCTTCCTTTTGCCTCTT | 309 |

### Example 5

### Selection of siRNA Comprising High Efficiency in Human Liver Cancer Cell Lines (Hep3B and Huh-7 Cell Lines) and Measurement of Inhibition Concentration 50% (IC50)

The human liver cancer cell lines (Hep3B and Huh-7 cell lines) were transfected using the siRNAs comprising sense strand of the SEQ ID NOs. 4, 118 and 204 selected in Examples 4-3-2, and expression levels of the target gene in the transfected human liver cancer cell lines (Hep3B and Huh-7 cell lines) were analyzed, thereby selecting the siRNA comprising the high efficiency. Then, performance of the siRNA was confirmed by measuring IC50 of the siRNA comprising the highest efficiency.

### Example 5-1

### Culture of Human Liver Cancer Cell Lines

The human liver cancer cell lines (Hep3B and Huh-7 cell lines) obtained from American Type Culture Collection (ATCC) were cultured under the same condition as that in Example 4-1.

The human liver cancer cell lines (Huh-7 cell lines) obtained from Korean Cell Line Bank (KCLB) were cultured in an RPMI-1640 culture medium (GIBCO/Invitrogen, USA) supplemented with 10% (v/v) fetal bovine serum, 100 units/ml penicillin, and 100 μg/ml streptomycin at 37° C. under 5% (v/v) CO₂ atmosphere.

### Example 5-2

### Transfection of the Desired siRNA in Human Liver Cancer Cell Lines

After the Hep3B cell lines cultured in Example 5-1 were cultured under the same condition as that in Example 4-2, 1.5 μl of Lipofectamine™ RNAi Max (Invitrogen, US) and 248.5 μl of the Opti-MEM medium were mixed with each other to prepare a mixed solution and then reacted with each other at room temperature for 5 minutes. Then, 0.8 or 4 μl of each of the siRNAs (1 pmole/μl), 0.2, 1, 5 or 20 μl of each of the siRNAs comprising sense strand of the SEQ ID NOs. 4, 118, 204 and 301 prepared in was added to 230 μl of the Opti-MEM medium, thereby preparing a siRNA solution comprising a final concentration of 8 pM, 40 pM, 0.2 nM, 1 nM or 20 nM.

The Lipofectamine™ RNAi Max mixed solution and the siRNA solution were mixed and reacted with each other at room temperature for 20 minutes, thereby preparing a solution for transfection.

In addition, Huh-7 cell lines cultured in Example 5-1 were cultured under the same condition as that in Example 4-2, 1.5 μl of Lipofectamine™ RNAi Max (Invitrogen, US) and 248.5 μl of the Opti-MEM medium were mixed with each other to prepare a mixed solution and then reacted with each other at room temperature for 5 minutes. Then, 0.8 or 4 μl of each of the siRNAs (1 pmole/μl), 0.2, 1, or 5 μl of each of the siRNAs comprising sense strand of the SEQ ID NOs. 4, 118, 204 and 301 prepared in was added to 230 μl of the Opti-MEM medium, thereby preparing a siRNA solution comprising a final concentration of 8 pM, 40 pM, 0.2 nM, 1 nM or 20 nM.

The Lipofectamine™ RNAi Max mixed solution and the siRNA solution were mixed and reacted with each other at room temperature for 20 minutes, thereby preparing a solution for transfection.

Thereafter, 500 μl of the solution for transfection was dispensed in each well containing tumor cell lines and the dispensed Opti-MEM medium and cultured for 6 hours, followed by removal of the Opti-MEM medium. Here, 1 ml of the RPMI 1640 medium was dispensed in each well and cultured at 37° C. under 5% (v/v) CO₂ atmosphere for 24 hours.

Total RNA was extracted from the cell lines transfected to prepare cDNA, and then a target gene mRNA expression level was relatively quantified using a real-time PCR by the same method as that in Example 4-3.

### Example 5-4

### Measurement of IC50

One kind of siRNAs was selected from the high efficiency siRNAs confirmed in Example 5-3 with respect to each of the genes, and performance of the corresponding siRNA was confirmed by confirming an IC50. After the Hep3B cell lines cultured in Example 5-1 were cultured under the same condition as that in Example 4-2, 1.5 μl of Lipofectamine™ RNAi Max (Invitrogen, US) and 248.5 μl of the Opti-MEM medium were mixed with each other to prepare a mixed solution and reacted with each other at room temperature for 5 minutes. Then, 0.8 or 0.4 μl of each of the siRNAs (0.01 pmole/μl) of the SEQ ID NOs. 1, 102, and 201 prepared in Example 1 or 0.2, 1, or 5 μl of each of the siRNAs (1 pmole/μl) comprising sense strand of the SEQ ID NOs. 1, 102, and 201 was added to 230 μl of the Opti-MEM medium, thereby preparing a siRNA solution comprising a final concentration of 8 pM, 40 pM, 0.2 nM, 1 nM, or 5 nM. The Lipofectamine™ RNAi Max mixed solution and the siRNA solution were mixed and reacted with each other at room temperature for 20 minutes, thereby preparing a solution for transfection.

In addition, after 1×10⁵ Huh-7 cell lines cultured in the Example 5-1 were cultured in a 12-well plate using the RPMI-1640 culture medium at 37° C. under 5% (v/v) CO₂ atmosphere for 18 hours, the medium was removed, and then 500 μl of the Opti-MEM medium (GIBCO, US) was dispensed in each well. Meanwhile, 1.5 μl of Lipofectamine™ RNAi Max (Invitrogen, US) and 248.5 μl of the Opti-MEM medium were mixed with each other to prepare a mixed solution and then reacted with each other at room temperature for 5 minutes. Then, 0.8 or 0.4 μl of each of the siRNAs (0.01 pmole/μl) comprising sense strand of the SEQ ID NOs. 1, 102, and 201 prepared in Example 1 or 0.2, 1, or 5 μl of each of the siRNAs (1 pmole/μl) comprising sense strand of the SEQ ID NOs. 1, 102, and 201 was added to 230 μl of the Opti-MEM medium, thereby preparing a siRNA solution comprising a final concentration of 8 pM, 40 pM, 0.2 nM, 1 nM, or 5 nM. The Lipofectamine™ RNAi Max mixed solution and the siRNA solution were mixed and then reacted with each other at room temperature for 20 minutes, thereby preparing a solution for transfection.

Thereafter, 500 μl of the solution for transfection was dispensed in each well containing tumor cell lines and the dispensed Opti-MEM medium and cultured for 6 hours, followed by removal of the Opti-MEM medium. Here, 1 ml of the RPMI 1640 culture medium was dispensed in each well and cultured at 37° C. under 5% (v/v) CO₂ atmosphere for 24 hours.

Total RNA was extracted from the transfected cell lines to prepare cDNA, and then a target gene mRNA expression level was relatively quantified using a real-time PCR by the same method as that in Example 4-3 (FIGS. 9A and 9B).

It was observed that the IC50 of the siRNA comprising sense strand of SEQ ID NO. 4 was 1 to 5 nM in the Hep3B cell lines and 0.2 to 1 nM in the Huh-7 cell lines (FIG. 5)

IC50 of the siRNA comprising sense strand of SEQ ID NO. 118 was 1 to 5 nM in the Hep3B cell lines and 0.2 to 1 nM in the Huh-7 cell lines (FIG. 6)

and IC50 of the siRNA comprising sense strand of SEQ ID NO. 204 was 8 to 40 pM in the Hep3B cell lines and 40 pM to 0.2 nM in the Huh-7 cell lines (FIG. 7)

Therefore, it was confirmed that the siRNA selected in the present invention had high efficiency.

### Example 6

### Confirmation of Cell Growth Inhibition by ZBTB7A, YAP1 or CHD1L Specific siRNA

Cells were transfected with a combination of the high efficiency siRNAs comprising SEQ ID NOs. 4, 118 and 204 as a sense strand confirmed in Example 4-3-2 at a concentration of 5, 20 and 100 nM, which was a concentration higher than the IC50.

1×10⁵ Huh-7 cell lines cultured in the Example 5-1 were cultured in a 12-well plate using the RPMI-1640 culture medium at 37° C. under 5% (v/v) CO₂ atmosphere for 18 hours, the medium was removed, and then 500 μl of the Opti-MEM medium (GIBCO. US) was dispensed in each well. Meanwhile, 1.5 μl of Lipofectamine™ RNAi Max (Invitrogen, US) and 248.5 μl of the Opti-MEM medium were mixed with each other to prepare a mixed solution and reacted with each other at room temperature for 5 minutes. Then, 5 μl of each of the siRNAs (1 pmole/μl) comprising sense strand of the SEQ ID NOs. 4, 118, 204 and 301 prepared in Example 1 was added to 230 μl of the Opti-MEM medium, thereby preparing a siRNA solution comprising a final concentration of 5 nM. The Lipofectamine™ RNAi Max mixed solution and the siRNA solution were mixed and reacted with each other at room temperature for 20 minutes, thereby preparing a solution for transfection.

Thereafter, 500 μl of the solution for transfection was dispensed in each well containing tumor cell lines and the dispensed Opti-MEM medium and cultured for 6 hours, followed by removal of the Opti-MEM medium. Here, 1 ml of the RPMI 1640 culture medium was dispensed in each well and cultured at 37° C. under 5% (v/v) CO₂ atmosphere for 24 hours.

Cell viability was confirmed by comparing the number of cells with that in the Experimental group treated with the siRNA comprising sense strand of SEQ ID NO. 301 (FIG. 12).

It may be confirmed that in the case in which the cell lines treated with the siRNAs of the SEQ ID NO. 4, 118 or 204, cell viability was concentration-dependently decreased, and the growth suppression effect was excellent (FIG. 8).

### Example 7

### Colony Forming Assay for Confirming Inhibition Effect of ZBTB7A, YAP1 or CHD1L Specific siRNA

A method of measuring transformation of cells by performing a colony forming assay on a single cell in vitro is a semi-quantitative method and is derived from lost of contact inhibition by the cancer cell and anchorage independent phenotypic characterizations of the cancer cell. This assay method is used to confirm survival of cancer cells by a specific anticancer drug in vitro in the case in which the cancer cells were treated with the corresponding anticancer drug (Clonogenic Assay of Cells in Vitro, Nat. Protoc. 1(5): 2315-9, 2006).

In order to confirm how much colony forming of the cancer cells was inhibited by the high efficiency ZBTB7A, YAP1 or CHD1L specific siRNA selected in Example 4-3-2, the colony forming assay (CFA) was performed. The hep3B and Huh-7 cell lines cultured in Example 5-1 were inoculated in a 35 mm Petri-dish (1×10⁴/dish), respectively. After 20 hours, the cells were transfected at a concentration of 5 nM or 20 nM by the same method as that in Example 5-2. The culture medium of the transfected cells was replaced once every three days, and after 10 to 14 days of the transfection, the cells were stained with Diff Quik (Sysmex, Japan) to compare colony forming degrees with each other (FIG. 9). It may be confirmed that in groups treated with the siRNAs comprising SEQ ID NO. 1 and 118 as a sense strand, colonies were concentration-dependently formed at a significantly low level as compared to the control group treated with the siRNA comprising sense strand of SEQ ID NO. 301 (FIG. 9).

1. A ZBTB7A, YAP1 or CHD1L specific siRNA comprising a sense strand comprising any one sequence selected from SEQ ID NOs. 1 to 300 and an antisense strand comprising a sequence complementary thereto.

2. The siRNA of claim 1, wherein the sense or antisense strand of the siRNA is composed of 19 to 31 nucleotides.

3. The siRNA of claim 1, wherein it is composed of a sense strand comprising any one sequence selected from a group consisting of SEQ ID NOs. 1, 2, 3, 4, 5, 6, 7, 8, 103, 107, 108, 112, 116, 117, 118, 121, 122, 201, 202, 203 and 204, and an antisense strand comprising a sequence complementary thereto.

4. The siRNA of claim 1, wherein the sense or antisense strand of the siRNA includes at least one chemical modification.

5. The siRNA of claim 4, wherein the chemical modification is at least one selected from modification by substitution of —OH group with —CH₃ (methyl), —OCH3 (methoxy), —NH₂, —F (fluorine), —O-2-methoxyethyl, —O-propyl, —O-2-methylthioethyl, —O-3-aminopropyl, —O-3-dimethylaminopropyl, —O—N-methylacetamido, or —O-dimethylamidooxyethyl at a 2′-carbon site of a sugar structure in a nucleotide;
modification by substitution of oxygen in a sugar structure in the nucleotide with sulfur;
modification of a nucleotide bond into a phosphorothioate bond, a boranophosphate bond, or a methyl phosphonate bond; and
modification into a peptide nucleic acid (PNA) type, a locked nucleic acid (LNA) type, or a unlocked nucleic acid (UNA) type.

6. The siRNA of claim 1, wherein at least one phosphate group(s) is bound to a 5′-end of the antisense strand of the siRNA.

7. Double-stranded oligo RNA molecule(s) comprising a structure of the following Structural Formula (1).
A-X—R—Y—B Structural Formula (1)
where A is a hydrophilic compound, B is a hydrophobic compound, X and Y each are independently a simple covalent bond or a linker-mediated covalent bond, and R is ZBTB7A, YAP1 or CHD1L specific siRNA.

8. The double-stranded oligo RNA molecule(s) of claim 7, having a structure of Structural Formula (2).
A-X—S—Y—B
AS Structural Formula(2)
where S is the sense strand of the siRNA of claim 7, AS is the antisense strand thereof, and A, B, X, and Y have the same definitions as those in claim 7.

9. The double-stranded oligo RNA molecule(s) of claim 8, having a structure of Structural Formula (3).
A-X-5′S3′-Y—B
AS Structural Formula (3)
where A, B, X, Y, S, and AS have the same definitions as those in claim 8, and 5′ and 3′ mean a 5′-end and a 3′-end of the sense strand of the siRNA, respectively.

10. The double-stranded oligo RNA molecule(s) of claim 7, wherein the ZBTB7A, YAP1 or CHD1L specific siRNA is the siRNA of claim 1.

11. The double-stranded oligo RNA molecule(s) of claim 7, wherein the hydrophilic compound has a molecular weight of 200 to 10,000.

12. The double-stranded oligo RNA molecule(s) of claim 11, wherein the hydrophilic compound is any one selected from a group consisting of polyethylene glycol (PEG), polyvinyl pyrrolidone, and polyoxazoline.

13. The double-stranded oligo RNA molecule(s) of claim 7, wherein the hydrophobic compound has a molecular weight of 250 to 1,000.

14. The double-stranded oligo RNA molecule(s) of claim 13, wherein the hydrophobic compound is any one selected from a group consisting of a steroid derivative, a glyceride derivative, glycerol ether, polypropylene glycol, saturated or unsaturated C₁₂-C₅₀ hydrocarbon, diacylphosphatidylcholine, fatty acid, phospholipid, and lipopolyamine.

15. The double-stranded oligo RNA molecule(s) of claim 14, wherein the steroid derivative is selected from a group consisting of cholesterol, cholestanol, cholic acid, cholesteryl formate, cholestanyl formate, and cholesteryl amine.

16. The double-stranded oligo RNA molecule(s) of claim 14, wherein the glyceride derivative is selected from mono-, di-, and tri-glycerides.

17. The double-stranded oligo RNA molecule(s) of claim 7, wherein the covalent bond represented by X and Y is a non-degradable bond or a degradable bond.

18. The double-stranded oligo RNA molecule(s) of claim 17, wherein the non-degradable bond is an amide bond or a phosphate bond.

19. The double-stranded oligo RNA molecule(s) of claim 17, wherein the degradable bond is a disulfide bond, an acid-degradable bond, an ester bond, an anhydride bond, a biodegradable bond, or an enzyme-degradable bond.

20. The double-stranded oligo RNA molecule(s) of claim 7, wherein a ligand which binds to a receptor promoting internalization into target cells through receptor-mediated endocytosis (REM) is additionally bound to the hydrophilic compound.

21. The double-stranded oligo RNA molecule(s) of claim 20, wherein the ligand is selected from a group consisting of a target receptor-specific antibody, aptamer, peptide, folate, N-acetyl galactosamine (NAG), glucose, and mannose.

22. Nanoparticle(s) comprising the double-stranded oligo RNA molecule(s) of claim 7.

23. The nanoparticle(s) of claim 22, composed by mixing double-stranded oligo RNA molecules containing siRNAs comprising different sequences with each other.

24. A pharmaceutical composition comprising the siRNA of claim 1, the double-stranded oligo RNA molecule(s) of claim 7, or the nanoparticle(s) of claim 22 as an active ingredient.

25. The pharmaceutical composition of claim 24, comprising a pharmaceutical composition for preventing or treating cancer.

26. The pharmaceutical composition of claim 25, wherein cancer is selected from a group consisting of liver cancer, gastric cancer, colon cancer, pancreatic cancer, prostate cancer, breast cancer, ovarian cancer, kidney cancer, and lung cancer.

27. The pharmaceutical composition of claim 26, wherein liver cancer is hepatocellular carcinoma (HCC).

28. Lypholized formulations comprising the pharmaceutical composition of claim 24.

29. A method for preventing or treating cancer characterized by administering a ZBTB7A, YAP1 or CHD1L specific siRNA comprising a sense strand comprising any one sequence selected from SEQ ID NOs. 1 to 300 and an antisense strand comprising a sequence complementary thereto of, the double-stranded oligo RNA molecule(s) of claim 7, or nanoparticle(s) comprising said double-stranded oligo RNA molecule(s), to an individual requiring such prevention or treatment of cancer.

30. The method for preventing or treating cancer of claim 29, wherein cancer is selected from a group consisting of liver cancer, gastric cancer, colon cancer, pancreatic cancer, prostate cancer, breast cancer, ovarian cancer, kidney cancer, and lung cancer.

31. The method for preventing or treating cancer of claim 30, wherein liver cancer is hepatocellular carcinoma (HCC).
